(19) United States
(12) Patent Application Publication
(10) Pub. No.: US 2019/0070371 A1
(43) Pub. Date: Mar. 7, 2019

(54) DEVICE FOR DISPENSING A SUBSTANCE, WHICH CAN BE DISCHARGED BY AIR

(71) Applicant: Alfred VON SCHUCKMANN, Kevelaer (DE)

(72) Inventor: Alfred VON SCHUCKMANN, Kevelaer (DE)

(21) Appl. No.: 16/178,870

(22) Filed: Nov. 2, 2018

Related U.S. Application Data
(63) Continuation of application No. PCT/EP2017/060398, filed on May 2, 2017.

(30) Foreign Application Priority Data
May 4, 2016 (DE) 10 2016 108 343.7
Aug. 23, 2016 (DE) 10 2016 115 568.3

(51) Int. Cl. A61M 15/00; A61M 11/02

(52) U.S. Cl. CPC A61M 15/0051; A61M 15/0043; B05B 11/064; A61M 2205/071; A61M 15/0053; A61M 11/02

(57) ABSTRACT

A device for dispensing a substance which can be discharged by air, comprising a discharge nozzle, a pump device, an air guiding channel joining the pump device to the discharge nozzle, a strip-shaped element which is mounted in the device and which comprises chambers which contains the substance and which are embodied successively over the entire length in the strip-shaped element, a reservoir and an emptying region, the strip-shaped element having several layers. The second layer of the chamber, which is located in the emptying region, is already removed in the partial region comprising said chamber and the air subjected to the forced flow flows through the air-guiding channel merging into the opening plane in the chamber, for advantageously emptying. Also, with respect to the pump device, the pump wall comprises a pressure region and several convex regions and when the convex regions are pressurised, the convex regions remain essentially the same and/or the device comprises a receiving region which is designed as a closed receiving chamber within the device, and that the partial region is located, during an anticipated movement, in the emptying region forming a movable part of the wall of the capture chamber.

### CROSS REFERENCE TO RELATED APPLICATIONS

This application is a continuation of and Applicant claims priority under 35 U.S.C. § 120 of International Application No. PCT/EP2017/060398 filed on May 2, 2017, which claims priority under 35 U.S.C. § 119 of German Application No. 10 2016 108 343.7 filed on May 4, 2016 and German Application No. 10 2016 115 568.3 filed on Aug. 23, 2016. The international application under PCT article 21(2) was not published in English. The disclosures of the aforesaid International Application and German application are incorporated by reference.

### BACKGROUND OF THE INVENTION

### 1. Field of the Invention

The invention relates to a device for dispensing a substance, which can be discharged by air. The invention in particular also relates to a device, which is also referred to as inhaler. It can serve a human user to take pharmaceutical substance by inhalation.

### 2. Description of the Related Art

Devices of the type in question are known. Reference is made in this respect for example to WO 2013/150021 A1 (US 2015/0114393 A1), WO 2008/037519 A1 (US 2010/0083962 A1) and WO 2004/009168 A1 (US 2008/0092885 A1). An inhaler is known from WO 01/26720 A1 (U.S. Pat. No. 6,880,555 B1), in the case of which a user, when he creates suction air, is supported by a forced flow created in the device.

The known devices are constructionally elaborate in part. They can further also not always be easily and safely operated in the desired manner.

A device for dispensing a substance, which can be discharged by air, is known from DE 100 11 120 A1, in the case of which a chamber is pierced with regard to its cover, in each case separately in the removal-ready position. A comparable prior art is also known from DE 10 2006 044 756 A1, US 2012/0090608 A1, US 2009/0229608 A1, US 2009/0151722 A1, and DE 10 2006 045 788 A1. Reference is to furthermore be made to DE 10 2006 044 756 A1, EP 2 077 132 A1, WO 2015/071667 A2, FR 1 051 645 A, and WO 90/07351 A1.

With regard to an embodiment of the pump wall, it is known from WO 2015/071667 A2 to embody a piston/cylinder unit, wherein the piston is to be displaced in a sealing manner relative to the cylinder in order to carry out a pump process. A closed pump bellows is known from FR 1 051 645 A.

With regard to an option of catching substance, which may not have been emptied, it is known from US 2009/0229608 A1 to move a strip element as a whole into a receiving chamber. A comparable prior art is also known from US 2009/0151722 A1.

With regard to a special embodiment of the pump wall, it is known from WO 90/07351 A1 to provide a piston-cylinder unit. A comparable embodiment is also known from WO 2015/071667 A2.

With regard to a further embodiment of the pump wall, reference is to also be made in this respect to WO 2015/071667 A2, which has already been mentioned.

### SUMMARY OF THE INVENTION

With regard to the cited prior art, the invention deals with the object of embodying a device for dispensing a substance, which can be discharged by air, in a favorable manner.

According to a first idea of the invention, the mentioned object is solved in the case of a device, which focuses on that, in an inhalation-ready position, the chamber is moved downstream from an air-permeable replacement cover.

The object is further solved in the case of a device, which focuses on that the pump wall is fastened to a housing of the device so as to form a seal, has a pressure region and several convex regions outside of the pressure region, and wherein the convex regions remain essentially undeformed in response to a movement of the pump wall from the initial position into the pump position.

The object is furthermore solved in the case of a device, which focuses on that the receiving region is set up as closed receiving chamber inside the device, a partial region of the strip-shaped element forms a movable part of a wall of the receiving chamber, and for this purpose, the strip-shaped element is guided so as to form a seal on the edge side so as to abut on corresponding stationary wall regions of the receiving chamber on the outer side.

The object is furthermore solved in the case of a device, which focuses on that a buckling of the pump wall takes place in a buckling convex region in response to a pump movement.

The object is finally solved in the case of a pump wall, which focuses on that the convex regions keep their form in response to a movement of the pump wall by impact of a force on the pressure region from the initial position into the pump position, in response to which the fastening edge can be unmoved.

In the context of the invention, the band element is also referred to as strip-shaped element. The strip-shaped element consists of a first and a second layer. In the context of the invention, the closure element is also referred to as second layer of the strip-shaped element.

According to a further idea of the invention, a device is specified for dispensing a substance, which can be discharged by air, comprising a discharge nozzle, through which air, which is loaded with substance can escape, and a pump device for creating air subjected to a forced flow, wherein an air guiding channel, which joins the pump device to the discharge nozzle is provided, through which air guiding channel air subjected to a forced flow can be guided, wherein the pump device for creating the forced flow further has a pump wall, which can be moved between an initial position and a pump position and which can be automatically returned from the pump position into the initial position, wherein the pump wall further has a fastening edge, a pressure region and several convex regions outside of the pressure region, and the convex regions remain essentially undeformed in response to a movement of the pump wall from the initial position into the pump position.

A pump volume, which can be used for emptying a chamber by flowing through the chamber, which lies between 6 ml and 15 ml, more preferably between 8 ml and 12 ml, beyond that more preferably at 10 ml, can be created by means of the pump device.

The idea of the invention can also be described to the effect that in the case of a device for the portioned dispensing of a powdery medicament, comprising a pump device for creating an air flow, the pump device can have an automatically returnable pump wall, wherein the pump wall can have several convex regions, which are formed from the pump wall itself, wherein the thickness of the pump wall is one-tenth or less, up to one-thousandths of a largest extension dimension of a convex region.

In its dimensions, the device described here is formed so as to be adapted in particular to the size of a palm of a hand. The pump device can be operated by a user, for example by means of the thumb.

The device can further be embodied for the portioned dispensing of a liquid or powdery medicament or a pharmaceutical substance, respectively, from chambers of a strip-shaped element to the effect that the strip-shaped element can be moved relative to a device housing, wherein the band element or the strip-shaped element, respectively, has continuous markings, one of which is in each case visible to the user through a window embodied in the device housing.

The device serves in particular for the mouth or nose inhalation of the medicament (pharmaceutical substance), which is present in portions in the chambers of the strip-shaped element. The substance is entrained by means of an air flow, which is activated in the course of the inhalation, i.e. an air flow subjected to a forced flow, and is discharged. Preferably, it is compressed air, which, in relation to a flow direction, is created upstream of a chamber, which is to be emptied, so that air is applied to the chamber at a higher pressure than corresponds to an ambient pressure, by means of being blown in.

Only this compressed air is preferably also used to empty a chamber. A user does not need to create a low pressure or needs to aspirate. The air, which is loaded with substance, is in fact blown into an oral cavity of a user when being used by means of the forced flow.

The chamber, which is moved into an emptying region, is preferably located completely exposed in the opening plane. In the opening plane, the air guiding channel connects to the chamber or the partial region, which has the chamber, respectively, in the emptying region. Provided that an air-permeable replacement cover is provided, see also further below, said air guiding channel can also connect to this air-permeable replacement cover or the latter can form a chamber-side mouth region of the air channel, respectively. It preferably does not protrude into the chamber located in the emptying region. Provided that a replacement cover is provided, it can form a chamber-side end region of the air guiding channel. With the exception that the second layer is removed, the chamber located in the emptying region is moreover preferably also intact, in particular not damaged with regard to the chamber wall by means of piercing means or the like.

The chamber wall of the individual chambers of the strip-shaped element is preferably formed of the strip material of the first layer, for example produced using the deep-drawing process. The chambers filled with the substance are initially closed by means of the second layer by placing the two layers against one another in a laminate-like manner. The second layer thus forms a closure element. The latter can also, and as is preferred, be embodied in a band-like manner in the form of a closure strip, which can be removed successively, and which is preferably cohesively integral and is more preferably also present integrally in the removed state in this way, according to the embodiment of the chambers, which are mounted successively.

A chamber preferably has an elongated trough-shaped form. A longitudinal axis, which corresponds to its direction of extension, preferably runs transversely to a longitudinal direction of the strip-shaped element.

A chamber can have a larger length than width. With regard to this, a ratio of length to width of 2:1 to 5:1, further for example approximately 3:1 can be present.

In a possible embodiment, the length of a chamber is directed transversely to a length of the strip-shaped element. The length of the strip-shaped element corresponds to a multiple of the width of a chamber. 10 to 50, further for example approximately 30 chambers of this type can be arranged on a strip-shaped element in the longitudinal extension of the strip-shaped element at a distance to one another, preferably at an even distance to one another. As a whole, the strip-shaped element, viewed in the use position in a layout position of the device, in which a geometrical axis of the device is depicted in a punctiform manner, can from an open circular ring comprising ends facing one another, which, in the use position in the device, have a distance of preferably less than one chamber extension in the circumferential direction. A strip-shaped element can for example have a width in the range of 2 to 10 mm, more preferably in the range of 4 to 8 mm.

In contrast to the support part (first layer), which forms the chambers, the closure element, which is preferably present in a band-like manner as second layer, can have a larger length, for offering a handling section, preferably for a corresponding seizing section in the device, at which this handling section can be held, whereby a peeling-like removal of the second layer from the first layer can be achieved in response to an operation of the device.

To prepare the inhalation, the closure element or the corresponding section of the second layer (closure strip), respectively, is removed from the chamber, which is released for the inhalation. To prevent an escape, for example trickling of the chamber content, in particular of the substance, for example of a medicament, out of the chamber prior to carrying out the inhalation in such an inhalation-ready position of the chamber, when the chamber is located in the emptying region, the chamber is in the inhalation-ready position, in which it is located in the device in the emptying region, preferably moves downstream from an air-permeable replacement cover, which replacement cover, in preferred embodiment, is mounted so as to be fixed to the housing. In contrast, the strip-shaped element having the chambers can be moved relative to the housing, in particular successively, i.e. gradually, with every operation about a partial region of the strip-shaped element having a chamber.

The air permeability of the replacement cover can be reached by means of a corresponding perforation of a corresponding housing part or housing section, respectively. A replacement cover in grid form can further be present here, for example comprising cover sections, which run so as to intersect one another, for example further in a wire-like manner. Such cover sections can also run parallel to one another in the same direction so as to form slit-shaped passages.

In the alternative, the replacement cover can also leave only 2,3 or at least few air passages, wherein one air passage is preferably assigned to a chamber beginning and one air passage is assigned to a chamber end.

The mesh size in the case of a grid-like, in particular cross grid-like structure of the replacement cover, as well as the width of the parallel distance of cover sections, which may run parallel alone, is selected in such a way that the in particular powdery substance can permeate this region not solely as a result of the force of gravity. In fact, an additional force component, in particular a forced air flow, which is also necessary for the inhalation, is mandatorily required for this purpose. By using this air flow, the chamber, which is brought into the ready position and which is freed from the closure element, can preferably be emptied completely, by penetration of the air-permeable replacement cover by the medicament or the substance, respectively, which is entrained by the air flow.

The chambers provided on the band element to as to be mounted successively with regard to the length of the band element are preferably designed of equal size among one another with regard to their receiving volume for the substance. A medicament dosage of virtually the same level results accordingly in response to every inhalation. An evacuation, which is as complete as possible, of the active chamber, i.e. which is located in the emptying position, is sought for this purpose. This evacuation preferably takes place only as a result of the air flow, which permeates the chamber. The air flow can be created by the user, as forced flow, with the help of a manual pump impact.

A pump wall is present on the device side, by means of which the forced air flow, preferably a compressed air flow, can be created in the device, for evacuating the activated chamber and for blowing out the air-substance mixture, for example air-medicament mixture, through the discharge nozzle. The forced air flow alone can be sufficient to carry out a proper inhalation. However, it can also be supported by an additional suction air flow, i.e. a suction pressure of the air, for instance as a result of the user breathing in.

The pump wall can have several convex regions, which, in preferred embodiment, are embodied in such a way that in response to a displacement of the pump wall to create the compressed air, a force threshold (which is preferably of the same level in the case of every inhalation process), has to be overcome initially. After overcoming this force threshold, only a smaller force is preferably still required to press down and thus blow the air through the chamber. In the context of the invention, this characteristic of the pump wall is also identified as collapse effect.

The user can accordingly only cause the pump operation, if he exerts a force, which is so high that the force threshold is overcome. A sudden pump movement results automatically hereafter, which is so strong that the created compressed air, thus the air, which is subjected to a forced flow, empties the chamber. Until a reaching of the force threshold, an air flow, which can create an effect with regard to an emptying of a chamber, is preferably not yet created.

The air created by means of the pump device, which is subjected to the forced flow, thus the compressed air, flows through the air guiding channel and the chamber, which forms a partial region of the air guiding channel in the emptying station and which is ready for emptying, and then further through the discharge nozzle. In response to a returning of the pump wall, air is sucked in from the outside again in reverse flow direction in the same way in a first embodiment. A separate air inlet is not provided according to the first embodiment.

According to a second embodiment, a separate airway is provided for the air, which is to be aspirated. The second embodiment is preferably also combined with a valve in the air channel, suitably a non-return valve, which is to be permeated by the air flow in response to a pump operation. The valve is preferably mounted in the air channel in a flow direction upstream of the chamber, which is located in the emptying position for being emptied.

Due to the fact that a comparatively large amount of air is created in response to a pump operation, a respective complete evacuation of the chamber located in the emptying region results, preferably also as a result of the described invasion effect, so that the use of this airway can readily also be used for the air inlet in response to the returning of the pump wall.

More preferably, a sudden compressed air flow, which starts with approximately the same pressure level, for evacuating the chamber thus results in response to each inhalation or in response to each chamber emptying, respectively.

In preferred embodiment, the thickness of the pump wall is 0.4 to 0.8 mm, more preferably approximately 0.6 mm. The pump part structured by the convex regions, the pump wall, preferably consists of a hard plastic, for example polycarbonate. As a result of the combination of the selected material, the thickness of the pump wall and embodiment of the convex regions, a pump wall, which can automatically be returned into the initial position, with invasion effect, is at hand. For a returning after operation of the pump wall, a separate spring or spring embodiment is thus not necessary. As a result of its embodiment, the pump wall can be returned automatically from the pump position into the initial position, without requiring further help.

The pump wall can have a fastening edge, by means of which it is fastened to the housing of the device so as to form a seal, and a pressure region, which can be moved for carrying out a pump process by reducing an air volume located between the pump wall and the device housing. The air volume, which can be discharged at least partially in a surge-like manner in response to the pump movement, thus preferably results accordingly between a cover-like housing region and the pump wall.

A convex region of the pump wall can hereby be embodied in an elongated manner, wherein the longitudinal direction can run from the fastening edge in the direction of the pressure region. In the case of an exemplary circular course of the fastening edge of the device, a substantially radial orientation of the longitudinal direction of the convex region—in relation to this course—can thus result. In this case, and more preferably also in the case of other layout forms of the device or of the pump wall fastening edge, respectively, the pressure region lends itself in the center with regard to the layout.

In a cross section, the fastening edge also preferably runs at an angle to the pressure region. In relation to an extension of the pressure region, an obtuse or right angle, which is drawn between the fastening edge and the pressure edge is preferred. The angle can extend for example between 80 and 120°, preferably approx. 90°.

In response to a pump movement, a convex region can be uninfluenced with respect to its length and width. Different convex regions can also be provided. In addition to the already-described convex region, which is virtually undeformed in response to an operation, one or several buckling convex regions can be provided.

The convex regions and the buckling convex regions can also be provided so as to be mounted side by side. In particular in such a way that a buckling convex region is embodied for the end region of a convex region, which is assigned to the pressure region. More preferably, this buckling convex region can have a significantly smaller length than the initially described convex region in a direction—viewed from the fastening edge to the pressure region.

In the case of an exemplary, as well as preferred, basic contour of the pump wall, which is circular with respect to its layout, several such convex regions are provided, which are preferably oriented radially, preferably spaced apart evenly to one another in the circumferential direction. Viewed over the circumference, six or more, up to thirty such convex regions can thus be provided.

Viewed in a circumferential direction, in the circumferential direction to the pressure region, rib-like transition regions can be embodied between two convex regions, which extend between the fastening edge and the pressure region. Such a transition region can have a front face, which points towards the pressure region, and the pressure region can have an outer edge, which is located opposite the front face and runs in the same direction. In the case of a mounting of the device, in which a force application onto the pressure region takes place in the perpendicular direction, the front face and the outer edge run in a cross section, in which the connecting axis appears as a line, preferably approximately vertically or at an acute angle to a vertical or the device axis, respectively.

A buckling convex region is preferably embodied between the front face and the outer edge, wherein the front face and the outer edge can also be viewed when being retracted into the buckling convex region. In the operating state, however, the buckling results virtually only in a convex base of such a buckling convex region. The front face and the outer edge are preferably moved towards one another so as to buckle, in a virtually inherently undeformed manner.

In a pump position, a buckling of the pump wall can now be present between the mentioned front face and the outer edge. In particular with regard to the preferably selected hard plastic, it can also happen that a stress whitening results in the buckling region during the course of several, for instance a plurality of pump movements.

Such a buckling can result in the described invasion effect in such a buckling, thus the transition from a relatively strong introduction of force into the pump wall and the preferably initially small movement thereof into a preferably stronger movement of the pump wall, which requires only a smaller application of force for itself. The transition from the smaller into the stronger movement of the pump wall is at hand as a result of a force threshold, up to which force threshold the required force, which is to be applied by the user, increases initially.

A clicker-like exceeding of a force introduction threshold is at hand.

The convex regions, preferably with regard to both described types, are provided so as to be curved inwardly, further with regard to the above-described cross section perpendicular to the surface, as it is mentioned further above with regard to the fastening edge, according to a pump wall surface surrounding the convex regions and being directed outwardly, in a recessed manner, viewed from the outside in the direction of a device interior. The depth of each convex region, viewed to the surrounding surface of the pump wall, is approximately one-half, to one-hundredth of a largest extension dimension of a convex region.

The convex regions and buckling convex regions embodied successively or next to one another can more preferably also have different lengths, thus further in particular different lengths in the radial direction.

The buckling convex region can have a smaller length than the convex region, which may be farther away from the pressure region. A ratio is thus preferably provided, in the case of which the smaller length corresponds to one-fiftieth, to one-third of the length of the larger convex region.

In the case of convex regions mounted next to one another, the buckling convex region, which is shorter in the mentioned direction, can be mounted between two convex regions, which are longer in the mentioned direction.

A hard plastic can be differentiated from a soft plastic, for example with regard to a Shore hardness, for instance measured according to DIN 53505 of 2012. In this regard, the hard plastic can in particular have a Shore hardness, which is >60, further in particular >65, for example a Shore hardness in a range of 75 to 80.

To show to the user, how many inhalations have already been made or how many inhalations can still be made, respectively, a corresponding display can be provided. Due to the fact that the strip-shaped element in the proposed device moves relative to the stationary housing, it lends itself to provide the marking directly on the strip-shaped band element.

The marking can be present in the form of numerical information, but as a further alternative also in the form of a for example color display, which changes for example from a green region to a red region in the course of the finishing of the individual chambers of the band element.

In preferred embodiment, a window is embodied in the housing, through which the marking provided on the band element is visible. Accordingly, the window is stationary.

The markings can thus be attached, for example imprinted, to the side of the strip-shaped element facing away from the closure element, thus preferably the first layer. The markings can be attached to the chamber walls and/or between them on the strip-shaped element.

The invention is to also be seen in that, in the case of a device for dispensing a substance, which can be discharged by air, comprising an air inlet, a discharge nozzle and an air guiding channel, in the case of which a strip-shaped element comprising chambers is mounted in the device, and a reservoir, in which the strip-shaped element can initially be received prior to a use and with regard to the part of the strip-shaped element, which has not been used yet, an emptying region is provided, in which the strip-shaped element can be moved gradually by a partial region, in which a chamber is embodied, out of the reservoir in the direction of the length, for discharging substance from a chamber by means of air, which is subjected to the forced flow, in the case of which a receiving region is further provided, into which the partial region can be moved directly downstream from the emptying region in response to a following gradual movement, wherein the receiving region is set up as closed receiving chamber within the device and the partial region, which had been located in the emptying region in response to the preceding movement, forms a movable part of a wall of the receiving chamber, which cooperates with stationary parts of the wall of the receiving chamber so as to form a seal.

A possibility is created thereby to introduce a substance, which may still be located in a chamber downstream from the emptying region, into a region within the device, which is closed apart from that, so that a state, in which a larger amount of substance is inadvertently dispensed, cannot occur.

The receiving chamber preferably has a volume, which corresponds to a multiple of a chamber volume, for example a 5-fold, 10-fold, up to 20-fold volume of a chamber volume. The volume can also correspond to the combined chamber volume of all chambers of a strip-shaped element, which is located in the device.

With regard to the stationary walls, the receiving chamber can be formed by wall parts, which start at opposite housing parts and preferably engage with one another at least over a part of their extension in a circumferential direction of the receiving chamber in the direction of the device axis. Due to the fact that a chamber is to be moved to the receiving chamber with the help of the strip-shaped element in such a way that the content thereof can be received in the receiving chamber, the receiving chamber as such cannot be sealed hermetically. It is provided, however, that the strip-shaped element itself forms a part of the wall of the receiving chamber, in each case in relation to the partial region, which is then assigned to the receiving chamber. For this purpose, the strip-shaped element can be guided so as to bear on the outer side on corresponding stationary wall regions of the receiving chamber so as to form a seal on the edge side. For example, a rail-like guide can be provided for the strip-shaped element, which can provide the desired seal between the strip-shaped element and the stationary parts of the wall of the receiving chamber. It can also be sufficient, however, to guide the strip-shaped element in the radial direction, closely along the outer surface of a corresponding wall of the receiving chamber, for instance as a result of a small dimension of the free space, in which the strip-element moves. A thickened embodiment of a guide wall, against which the closed region of the chamber bears, is preferred. The thickened embodiment can be attained by means of a rib-like structure, which more preferably has a longitudinal extension in the direction of the longitudinal extension of the strip-shaped element. The thickened embodiment can also be a thickened wall over the substantially entire height, against which the corresponding rear side of the strip-shaped element, in which the chamber is closed, bears during the transport.

In the position, in which it is assigned to the receiving chamber, the chamber in the strip-shaped element can be exposed towards the interior of the receiving chamber in a window-like region of a stationary wall of the receiving chamber.

In this mounting of the chamber in relation to the receiving chamber, either a first or further emptying of the chamber into the receiving chamber can take place by means of force of gravity or a further blow-out of the chamber with air can for example also be provided, possibly by means of a branched-off air flow of the air, which is subjected to the forced flow. A first emptying can commence, when a user performs a double transport operation with regard to the strip element without inhaling after a first operation. In the alternative or in addition, mechanical means can also be mounted, for instance in the form of bristles, which sweep out the chamber, in the course of their movement into the position assigned to the receiving chamber and/or out of the position assigned to the receiving chamber into a further receiving region for the strip-shaped element, the chambers of which are emptied.

On the whole, a use of a strip-shaped element can be provided in response to a use of the device, which, in the top view, can in particular extend over at least approximately 360°, by adapting to the circular shape of the outer contour of the device. The strip-shaped element can then be further displaced successively, so that chamber after chamber is located in the emptying region.

In preferred embodiment, the device needs to be disposed of, together with the received strip-shaped element, possibly with separated or largely separated first and second layers. Preferably, it is a single-use device, which is to be used to finish a strip-shaped element, which is provided with chambers.

A flowing of air through the chamber for emptying purposes can take place, as is also preferred, in the direction of a longitudinal direction of the chamber. In the emptying-ready position, the chamber forms a part of the air channel, which can be flown through, The emptying is preferably achieved solely as a result of flushing with air.

The longitudinal chamber direction can, and as is preferred, run so as to be aligned with a direction of extension of a geometrical central axis of the device. This central axis can run so as to correspond or coincide with an axis of rotation, about which a gradual displacement of the band element can be carried out.

The invention also relates to a device for dispensing a substance, which can be discharged by air, wherein the device is provided with a device housing, an air inlet and a discharge nozzle, through which air, which is loaded with substance, can escape, and comprising an air guiding channel joining the air inlet with the discharge nozzle and through which air, which is subjected to a forced flow, can be guided, a pump device comprising a pump wall, which can be moved between an initial position and a pump position, for embodying the forced flow, wherein the device housing has a first and a second broad side, which are located opposite one another with outer surfaces formed so as to face away from one another and which are essentially congruent in a top view onto one of the broad sides and are spaced apart by a narrow side, wherein the first and the second broad side further have a corresponding smallest width dimension, which is measured along a line through a center of an area of the broad side, based on the top view, and the narrow side determines a thickness of the device housing, wherein the pump wall furthermore forms a part of the first broad side, the thickness is smaller than the smallest width dimension, and the discharge nozzle, viewed in the top view, appears as a formation of the broad side, which protrudes in a tip-shaped manner. Such a device, but also in one of the other further embodiments described here, is preferably dimensioned so as to be adapted to the size of a human hand.

The features, which are described separately with regard to the device, one, several or all of these features can be provided so as to be combined with the features of the initially described idea of the invention, and also with the features of the further idea of the invention, in the manner as already described above. Not all features need to be realized hereby in each case, one or several of the initially described or of the further idea of the invention or of the description of the device can be combined here.

In the case of a device for the portioned dispensing of a liquid or powdery medicament in response to a mouth or nose inhalation, such a device comprising a discharge nozzle and a pump device can be provided for creating an air flow, wherein the device has a device housing comprising a smaller thickness than width, and the discharge nozzle protrudes on a narrow side of the device housing, and a pump wall embodied for operation by the user is embodied on a broad side of the device housing.

The width of the device housing can correspond to 2-times to 5-times, more preferably approximately to 3-times the thickness of the device housing, viewed perpendicularly thereto. In the case of a possible circular layout of the device housing, the width corresponds to the diameter.

A narrow side of the device housing results accordingly in the thickness direction. This narrow side extends circumferentially to the housing layout. The discharge nozzle protrudes beyond this narrow side, thus protruding with respect to the other device housing, which essentially predetermines the layout, for favorably surrounding with the lips in response to an oral inhalation or for insertion into a nostril, respectively.

The pump wall of the pump device is embodied on a broad side of the reservoir housing and thus essentially perpendicular to the general orientation of the discharge nozzle.

As a result of a thickness dimension of the device housing, which is preferably chosen to be ergonomically favorable, the user can thus seize the device in the region of its housing between for example thumb and index and/or middle finger when the discharge nozzle points towards the user, and the inhalation process can be carried out in an ergonomically favorable manner by supporting the device housing on the thumb by pushing operation via the fingers on the pump wall. The device is preferably embodied for the one-handed operation, in particular with regard to the actual inhalation process.

The device can further, and also preferably, have a transport device, for transporting a chamber, which is filled with substance, into a discharge position, wherein an operable transport lever for a user protrudes on a narrow side of the device housing. This transport lever is also located in ergonomically favorable orientation and position.

The transport lever can thus further be mounted to the device housing so as to be located opposite to the discharge nozzle, thus on the rear of the device housing when looking at the device onto the discharge nozzle.

Viewed in the pressure direction of the pump wall, the device housing can have a basic circular contour, as is preferred, comprising discharge nozzle and transport lever protruding radially with respect thereto. Discharge nozzle and transport lever can thus further protrude radially beyond the circular basic contour of the device housing essentially in diametrically opposite regions.

In further as well as preferred embodiment, the transport lever can be surrounded by a boundary wall, which also protrudes radially beyond the circular basic contour. Said boundary wall can thus serve to protect the transport lever against unintentional operation. The boundary wall thus leaves a free space for operating the transport lever.

With regard to the disclosure, the ranges or value ranges, respectively, or multiple ranges specified above and below also include all intermediate values, in particular in 1/10 steps of the respective dimension, possibly also without dimension. For example, the information 1/10 to 1/1000 also includes the disclosure of 11/100 to 1/1000, 1/10 to 9/10000, 10/100 to 9/10000, etc., the disclosure of 1/50 to 1/5 also includes the disclosure of 11/500 to 1/5, 1/50 to 9/50, 11/500 to 9/50, etc. On the one hand, this disclosure can serve to limit a mentioned range limit from below and/or the top, but in the alternative or additionally, to disclose one or several singular values from a respectively specified range.

### BRIEF DESCRIPTION OF THE DRAWINGS

The invention is described below by means of the enclosed drawing, which, however, only represents an exemplary embodiment:

FIG. 1 shows a device for the portioned dispensing of a substance in perspective illustration, relating to a basic position;

FIG. 2 shows a view against a narrow side of the device;

FIG. 3 shows the top view onto a broad side of the device;

FIG. 4 shows a further side view of the device;

FIG. 5 shows the device in further side view;

FIG. 6 shows the side view against the narrow side of the device located opposite the narrow side illustrated in FIG. 2;

FIG. 7 shows the section according to the line VII-VII in FIG. 3;

FIG. 8 shows the magnification of the region VIII in FIG. 7;

FIG. 9 shows the device in a perspective exploded illustration;

FIG. 10 shows the device in perspective illustration by omitting the housing and a counter plate;

FIG. 11 shows a sectional illustration corresponding to FIG. 7 through the device; but relating thereto for the inhalation of the substance in the course of a chamber emptying;

FIG. 12 shows the magnification of the region XII in FIG. 11;

FIG. 13 shows a strip-shaped element comprising several chambers, covered by a closure element;

FIG. 14 shows an enlarged view of the element, relating to an outer view against the chambers;

FIG. 15 shows a replacement cover comprising a part of the air guiding channel in perspective illustration at an angle from the front (chamber side);

FIG. 15a shows the replacement cover according to FIG. 15 in a perspective view at an angle from the rear;

FIG. 16 shows the replacement cover according to FIG. 15 in a front view;

FIG. 17 shows a section through the replacement cover according to FIG. 15 or FIG. 16, respectively, along line XVII-XVII in FIG. 16;

FIG. 18 a transport wheel in view;

FIG. 19 shows the transport wheel in perspective illustration;

FIG. 20 shows a pump wall of the device in top view;

FIG. 21 shows the side view thereto;

FIG. 22 shows the magnification of the region XXII in FIG. 20;

FIG. 23 shows the enlarged section according to line XXIII-XXIII in FIG. 20;

FIG. 24 shows a view according to for instance FIG. 1, with a cutaway in the region of the receiving chamber;

FIG. 25 shows an enlarged view of the selected region according to FIG. 24;

FIG. 26 shows a top view according to FIG. 3, again with a cutaway in the region of the receiving chamber;

FIG. 27 shows a magnification of the region XXVII in FIG. 26;

FIG. 28 shows a sectional illustration of the device according to FIG. 26, cut along line XXVIII-XXVIII, with illustration of the air flow in response to a movement of the pump wall from the initial position into the pump position;

FIG. 29 shows an illustration according to FIG. 28, with an illustration of the air flow in response to a return movement of the pump wall into the initial position;

FIG. 30 shows a sectional illustration of the subject matter according to FIG. 26, cut along line XXX-XXX;

FIG. 31 shows a magnification according to FIG. 27, with removed housing cover, but also with illustration of the part of the air guiding channel illustrated hereby; and

FIG. 32 shows a cross section through FIG. 31, cut along line XXXII-XXXII in FIG. 31;

FIG. 33 shows a perspective sectional illustration of the mouthpiece-side region, with removed housing cover and pump wall;

FIG. 34 shows a perspective, partially cut open embodiment of the device comprising a non-return valve mounted in the air guiding channel and a separate aspiration path for air to be aspirated, in the case of an illustrated airway in response to an inhaling;

FIG. 35 shows a complete sectional illustration of the device according to FIG. 34, with illustrated airway in response to an exhaling;

FIG. 36 shows an enlargement of the region XXXVI in FIG. 34;

FIG. 37 shows an enlargement of the region XXXVII in FIG. 35;

FIG. 38 shows an enlargement of the region XXXVIII in FIG. 35;

FIG. 39 shows an illustration of the alternative way of inserting a screen in the air guiding channel;

FIG. 40 shows a further illustration of the device comprising an embodiment of the discharge nozzle as mouthpiece in the top view; and

FIG. 41 shows an illustration according to FIG. 40 in the side view.

### DETAILED DESCRIPTION OF THE PREFERRED EMBODIMENTS

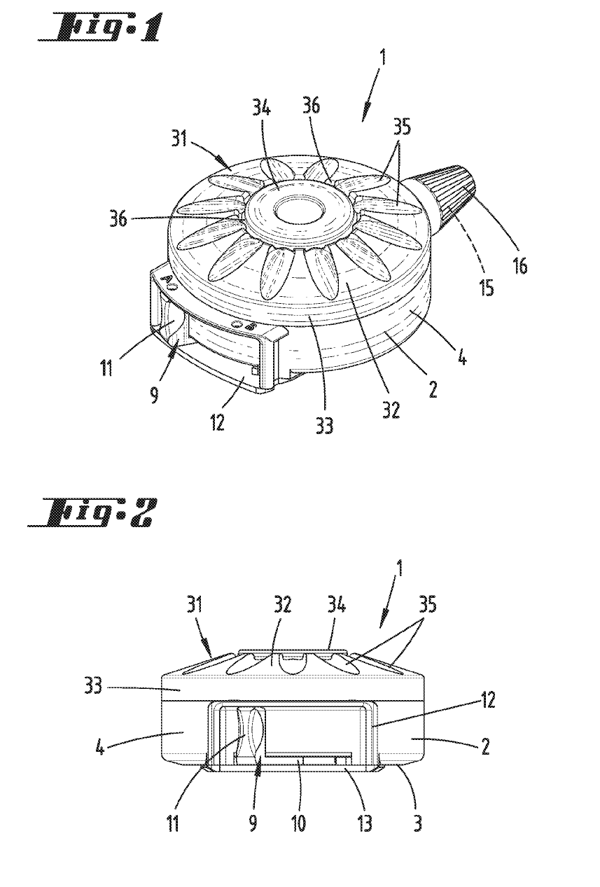

With reference to FIG. 1, what is initially illustrated and described is a device 1 for the portioned dispensing of a substance 27, in particular of a powdery medicament. The device 1 can serve for the oral and/or nasal inhalation.

A pot-shaped device housing 2 is provided, comprising a housing bottom 3 and a housing wall 4, which is circumferential in the sense of a pot wall. The device housing 2 determines form and, in the illustrated embodiment, has a, as well as preferably, substantially circular layout. The diameter and width d of the device housing 2 resulting therefrom corresponds approximately to 5-times the thickness e, viewed perpendicularly to the broad side.

In relation to FIG. 3, the width d is provided with regard to the broad side of the device, which is visible there. FIG. 3 represents a top view onto this broad side. As can be seen, the device also has two essentially congruent broad sides, on the one hand essentially provided by the housing bottom 3 and, on the other hand, essentially provided by the pump wall 32. These broad sides are also provided so as to point away from one another. The measure d is taken along a straight line through the center of area, wherein the center of area with regard to the broad side, which is provided in FIG. 3 (but also with regard to the opposite broad side) is provided by the device axis x, which is illustrated in a punctiform manner in this illustration.

A support wall 5 can be provided at a radial distance, offset inwardly to the housing wall 4, coaxially to the housing wall 4 and extending from the same broad side of the housing bottom 3. The free outer edge thereof, viewed in the axial direction, preferably ends in the same plane as the free outer edge of the housing wall 4.

A circumferential locking toothing 6, which is directed radially outwards, can further be embodied on the housing bottom 3 so as to be surrounded by the support wall 5 at a radial distance.

Receiving the device axis x centrally—and here accordingly also the housing axis—a bore 7 can be molded in the housing bottom 3. A joining pin 8 of a transport lever 9 can further dip from the outside axially inwards through said bore.

With regard to a lever arm 10, which is preferably part of the transport lever 9, the transport lever 9 can be embodied in a plate-shaped manner. The lever arm 10 can bear on the support wall 5. The lever arm 10 can extend radially outwards beyond the housing boundary, so as to form an operating section 11.

The operating section 11 preferably protrudes radially beyond the housing 2 on a narrow side of the reservoir housing 2, so as to be capable of being seized by a user in an advantageous manner. In the illustrated exemplary embodiment, and preferably, said operating section is furthermore surrounded by a boundary wall 12, which radially protrudes beyond the housing wall 4. The boundary wall 12 can be embodied integrally and of uniform material with the housing 2.

The transport lever 9 is held so as to be rotatable around the device axis x, wherein a pivot distance of preferably 15 to preferably 45°, further for example 30°, is also provided and limited by the boundary wall 12.

The surface of the lever arm 10 facing away from the housing bottom 3 is preferably covered by a counter plate 13, which can extend all the way to a circumferential outer edge of the boundary wall 12. The counter plate 13 can be interlocked with the housing 2.

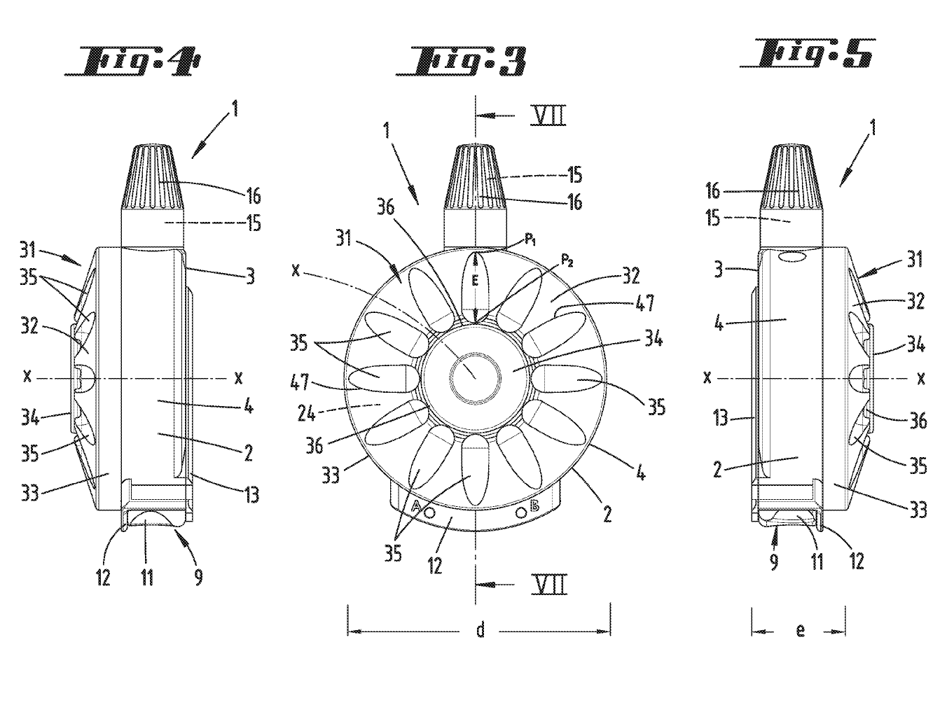

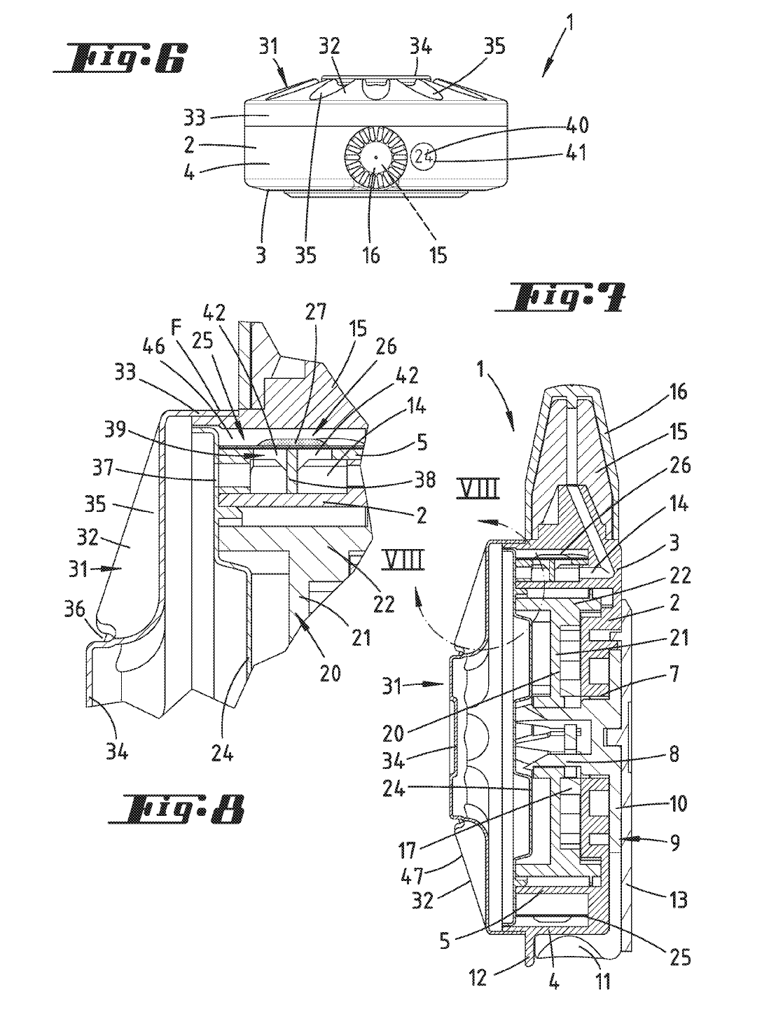

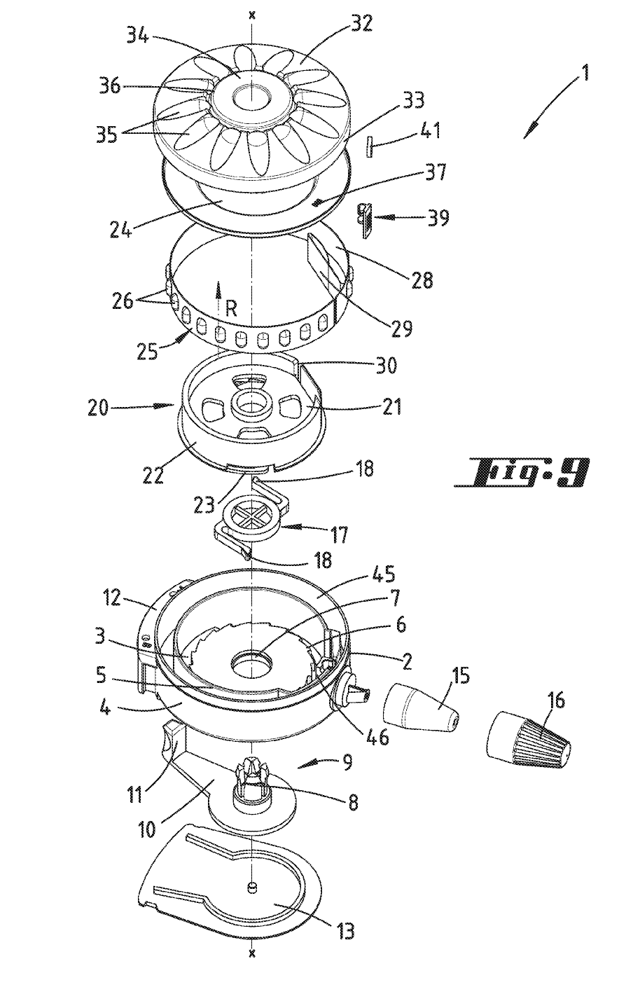

A boundary wall 12, which is circumferential in view against the narrow side of the device housing 2 according to FIG. 2, thus further results in a preferably manner.

Located essentially diametrically opposite the boundary wall 12, based on the device axis x, the housing wall 4 can be perforated by an air guiding channel 14. Air, which is subjected to a forced flow and which can preferably be created in a way as has been described above or will be described below, respectively, can be capable of being guided through the air guiding channel 14. The air guiding channel 14 can lead into a discharge nozzle 15, which protrudes freely radially outwards beyond the housing wall 4. In the case of non-use of the device 1, the discharge nozzle 15 can be covered by a cap 16 according to the illustrations. The perforation of the air guiding channel 14 and the reach-through of the lever arm 10 through the boundary wall 12 are preferably the only perforations or reach-throughs, respectively, of the boundary wall 12.

The transport lever 9 can be joined in a rotationally fixed manner to a follower 17 via the joining pin 8. The follower 17 preferably extends essentially in a plane, which is oriented transversely to the device axis x.

In the illustrated exemplary embodiment and preferably, the follower 17 has two spring arm-like latching fingers 18, which are essentially molded so as to be located diametrically opposite one another, for interaction with a toothed ring 19 of a transport wheel 20, which is oriented coaxially to the device axis x and which is held so as to be rotatable about said axis x.

The transport wheel 20 can have a bottom 21, which is circular disk-shaped in the exemplary embodiment, and a circumferential wall 22, which is preferably molded thereto. The bottom 21 can extend approximately centrically to the direction of extension of the wall 22 in the direction of the device axis x.

The toothed ring 19 is preferably molded on the bottom side of the bottom 21, facing the follower 17, wherein the teeth of the toothed ring 19, which are distributed evenly over the circumference, can leave a radial distance to the facing inner surface of the wall 22, into which free space for example the follower-side latching fingers 18 can dip.

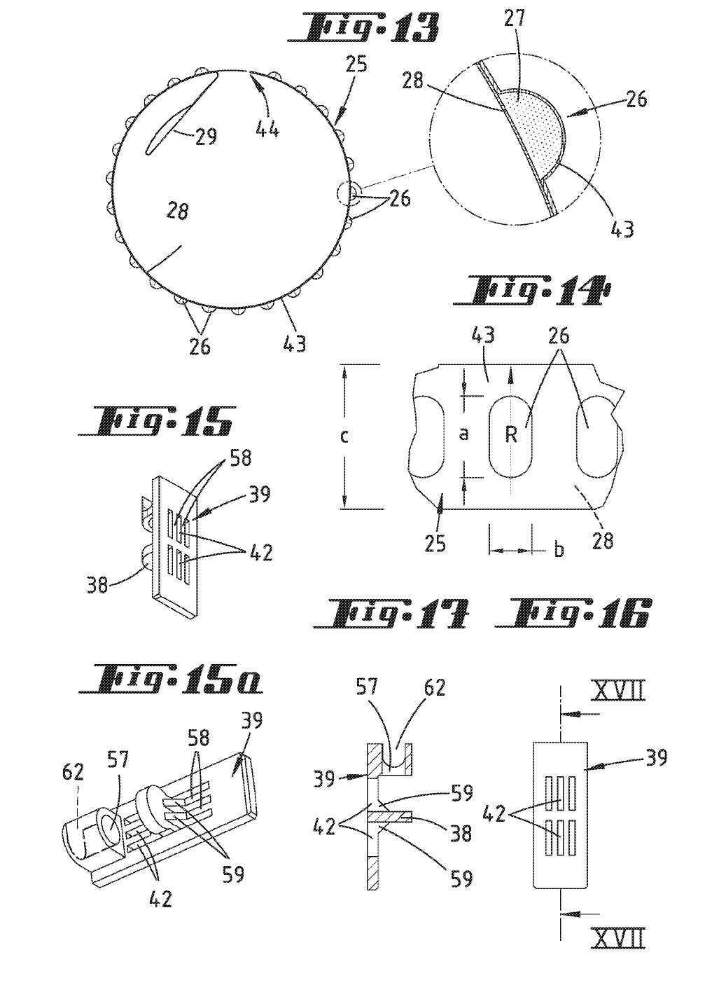

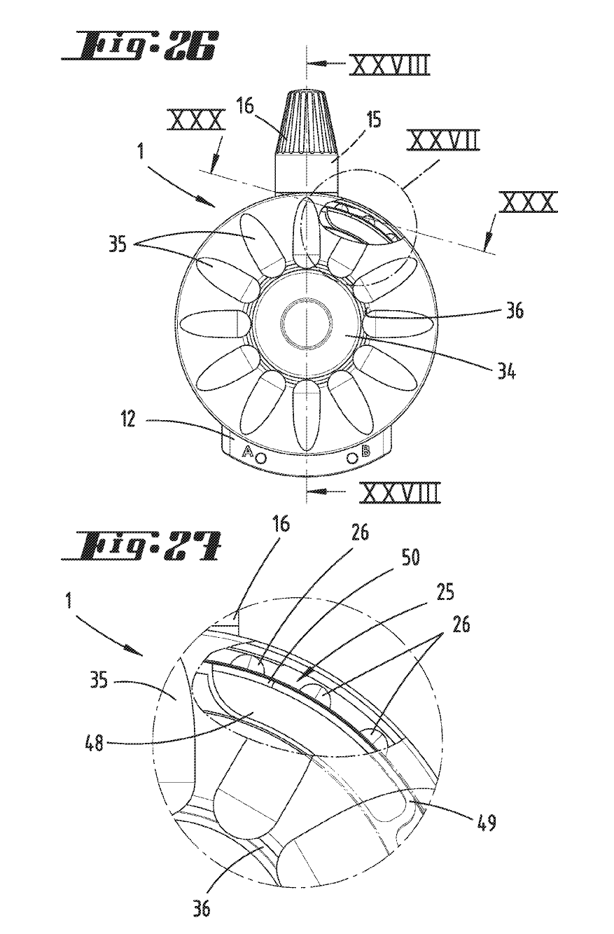

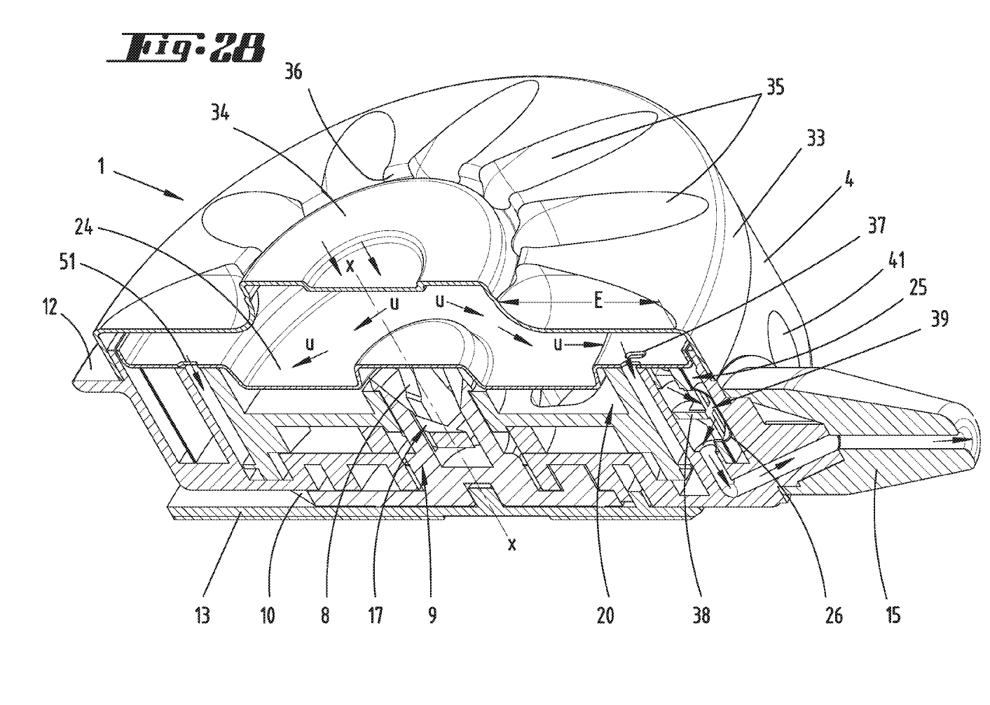

One and preferably two retaining fingers 23, which are cut free for the ability to deviate elastically, can be molded in the region of the wall 22 (see in particular FIGS. 10, 18 and 19). These retaining fingers 23—in the illustrated exemplary embodiment two retaining fingers 23 located diametrically opposite—interact with the housing-side locking toothing 6.

The two toothings between retaining finger 23 and locking toothing 6 as well as between latching finger 18 and toothed ring 19 are oriented in opposite directions, each with a predetermined entrainment direction and an overrun direction, which is opposite thereto in the direction of rotation, for the retaining fingers 23 or the latching fingers 18, respectively. When returning, the latching fingers 18 or the retaining finger 23, respectively, travel over their respective corresponding toothed rings as a result of elastic deviation.

A housing cover 24 is further provided. In the illustrated exemplary embodiment and preferably, said housing cover is provided with an essentially circular layout, preferably according to the circular design of the housing wall 4.

The housing cover 24 is joined to the housing wall 4 in a preferably non-releasable manner. The housing cover 24 can be interlocked with the housing wall 4, furthermore in the alternative for example also welded or adhered.

A film-like, strip-shaped element 25, which, in an adaptation to a circumferential outer contour of the housing, extends over almost 360°, is preferably received in the circumferential gap, which results between the inner side of the circumferential housing wall 4 and the outer side of the support wall 5 in the illustrated exemplary embodiment. The receptacle can be divided into a reservoir, an emptying region and a receiving region. In the course of a use of the device, the strip-shaped element can clear a reservoir for receiving a used region of the strip-shaped element by means of successive advancement. Chambers 26 are molded in the strip-shaped element. The strip-shaped element 25 has a length and a width c. The length is several times larger than the width c, for example 10- to 100-times larger.

More than 15 chambers 26, up to 30, 40 or 50 chambers, can be provided in the strip-shaped element, distributed over the length, with regard to the circular mounting also distributed over the circumference. An embodiment comprising 24 chambers 26 is illustrated. The chambers 26 are embodied so as to preferably be spaced apart evenly to one another over the circumference of the device or over the unwound length of the element 25, respectively, according to the displacement of the strip-shaped element, which is in each case provided by the possible pivot displacement distance of the transport lever 9. In a state of the element 25, in which it is inserted into the device, a chamber 26 has a longitudinal chamber direction R, which corresponds to the direction of the device axis x (axis of rotation).

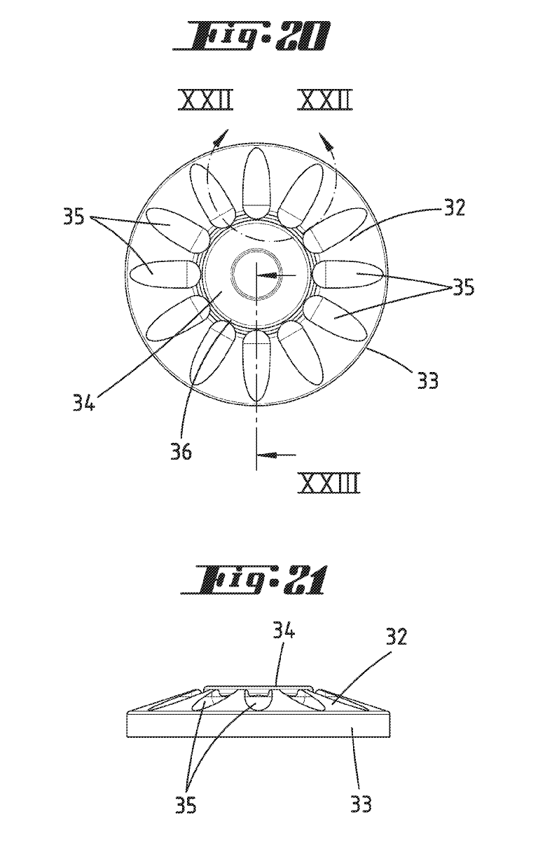

The chambers 26 have a larger length a than width b. A length a is thus provided, which corresponds approximately to 2-times the width b as viewed transversely thereto (see FIG. 14). The longitudinal orientation of each chamber 26 is directed transversely to the longitudinal extension of the strip-shaped element 25.

The width c of the element is preferably adapted to the axial free dimension in the circumferential gap region between housing wall 4 and support wall 5.

In an initial state, in which they are also located in the reservoir 45, the chambers 26 are filled. In the exemplary embodiment, they are preferably filled with a pharmaceutical substance, for example a powdery medicament. All chambers 26 preferably have the same volumes. The same doses of medicament are thus present.

The chambers 26, which are filled with the substance (medicament), are formed by a first layer 43 of the strip-shaped element 25 and are closed along an opening plane 0 by means of a band-like closure element 28, which forms a second layer 28 (see also enlarged illustration of FIG. 13 and FIG. 11 with regard to the opening plane 0). On the rear side of the opening plane 0, the chambers each form a chamber elevation 56. The second layer 28 thus extends facing away from the chamber elevations 56 along the facing first layer 43, covering the chamber openings, and is joined to the first layer 43. Originating from an element end 44, the second layer 28 extends, preferably starting at this element end 44, beyond the opposite element end, so as to provide a handling section 29. The handling section 29 can be caught in a slit formation 30, which can represent a seizing section of the device for the handling section 29. The slit formation 30 can be embodied in the transport wheel 20.

The first layer 43 can be joined to the second layer by means of an adhesive, which provides for the separation of the layers, in particular a pressure-sensitive adhesive.

The housing cover 24 is preferably used to form a pump device 31. For this purpose, said housing cover is preferably covered by a pump wall 32. The latter, as furthermore preferably all parts of the device 1, possibly with the exception of a valve, for example, is made of a hard plastic, but in particular as compared to the housing parts with a preferably smaller thickness f of approximately 0.2 to 0.8 mm, preferably approximately of 0.6 mm. A relatively thin-walled pump wall 32 is thus provided. If a screen is provided, said screen can possibly also be made of a metallic material.

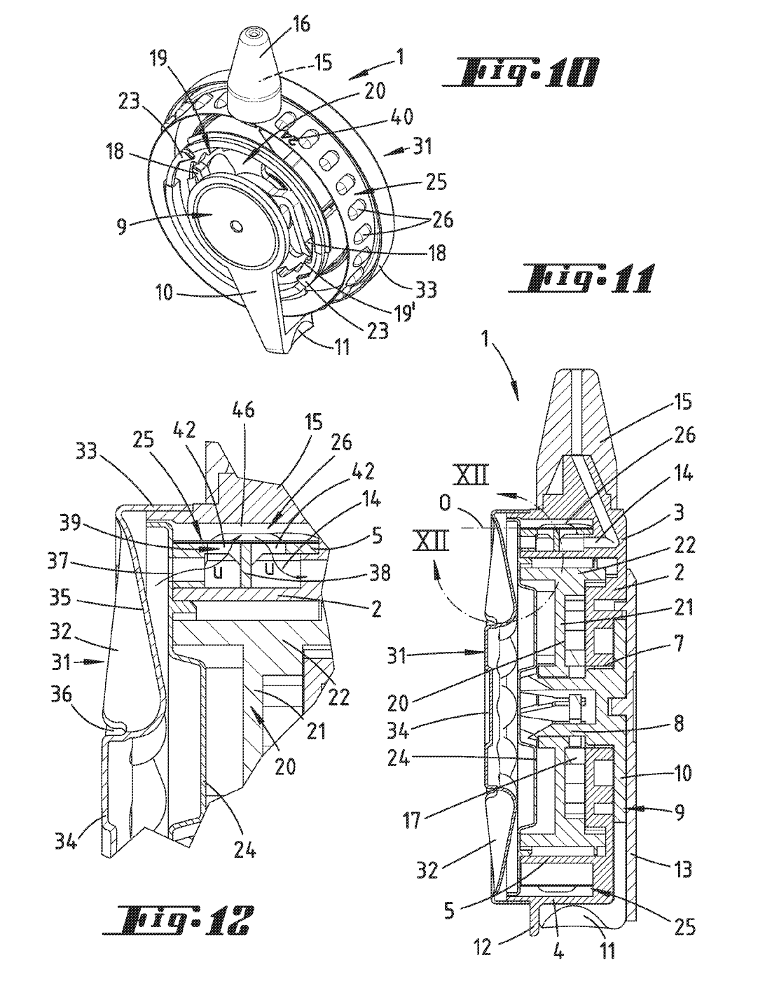

The pump wall 32 can have a circumferential fastening edge 33, by means of which the pump wall 32 is fastened to the device housing 2 so as to form a seal. Starting at the fastening edge 33, the pump wall 32 extends, in relation to a cross section perpendicular to the plane extension of the housing bottom 3 according to the illustration in FIG. 7, preferably in a bellows-like manner, hereby more preferably centrically, so as to accommodate the device axis x, forming a pressure region 34. In the uninfluenced basic position according to FIGS. 7 and 8, this pressure region can extend essentially directed transversely to the device axis x, more preferably essentially oriented parallel to the housing bottom 3. A housing-side end of the fastening edge 33, for instance in relation to the cross sectional illustration in FIG. 8, can also determine a surface F, perpendicularly to which a pump movement of the pump wall takes place.

The pump wall region, which extends from the pressure region 34 to the fastening edge 33, preferably has convex regions 35, 36. These convex regions 35, 36 can preferably be for example bead-like curvatures of the pump wall 32, which dip inwards.

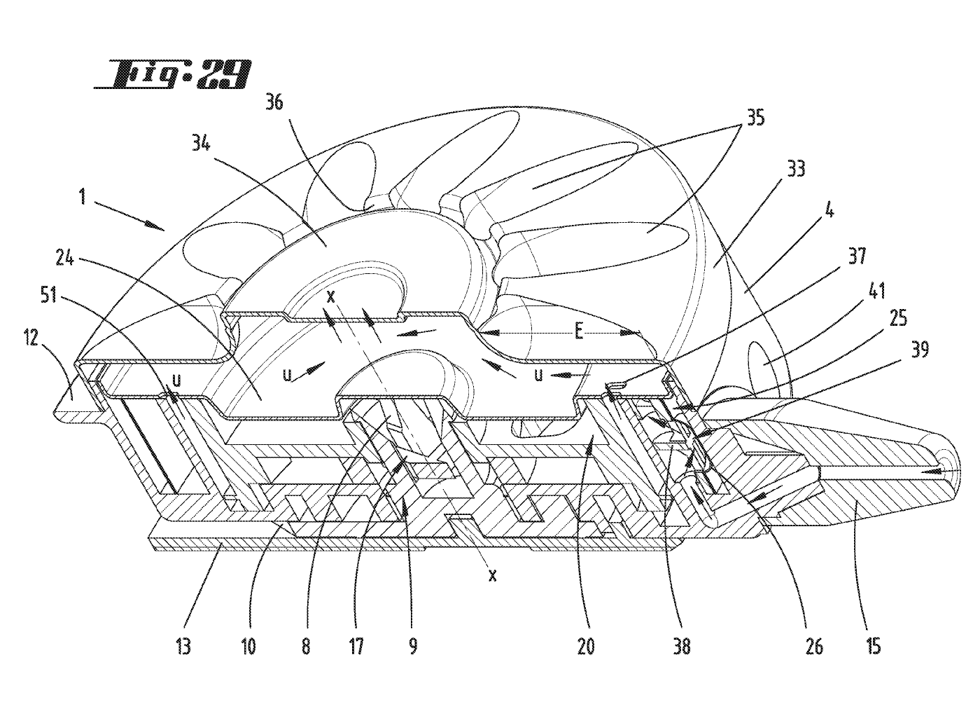

In relation to a plan view of the device, in which the device axis x is depicted in a punctiform manner, see FIG. 3, viewed in a circumferential direction, preferably two different convex regions 35 and 36 are in each case embodied next to one another, wherein one convex region 35 preferably has a length, which is larger as compared to the convex region located next to it in the circumferential direction, which can also be referred to as buckling convex region 36, more preferably a multiple length in the radial direction. A corresponding length g, see also FIG. 23, of the longer convex region 35 is thus provided, which corresponds approximately to 3- to 50-times, preferably approximately to 6- to 10-times, more preferably for instance to 8-times the corresponding radial length h of the shorter convex region 36.

The width m of the longer convex region 35, viewed transversely to the length g, can correspond approximately to 0.3- to 0.5-times the corresponding length, wherein, further starting at a radially inner end region, the width m decreases successively radially outwards towards the fastening edge 33.

In the illustrated exemplary embodiment, twelve longer convex regions 35 and twelve buckling convex regions 36 are provided so as to be distributed evenly over the circumference.

In the illustrated exemplary embodiment, a buckling convex region 36 is formed by a bead, which is embodied so as to be open on both sides in relation to the device axis x in a circumferential direction to the pressure region 34. In addition, the bead preferably has a bottom surface, which, viewed from the outside, is curved in a convex manner, and which transitions into the convex regions of adjacent longer and preferably also significantly broader convex regions 35.

In relation to a cross section according to FIG. 23, an elevation, which, preferably seen in an illustrated initial position of the pump wall 32, directed outwards from a convex base of the convex region 35, for instance in the form of a rib-like transition region 52 of the pump wall 32, can result in the transition between the convex region 35 and the bead-like buckling convex region 36. The pump position of the pump wall 32 is suggested in FIG. 23 with a dashed line. The pressure region 35 preferably moves from the initial position into the pump position, and more preferably from the pump position into the initial position, essentially parallel to itself.

Based on a direction from the fastening edge to the pressure region, the transition region 52 limits the buckling convex region 36 by means of a front face 53 and hereby also partially forms the buckling convex region 36. The front face 53 extends essentially parallel or at an acute angle to the device axis x, in a cross section, in which the device axis is depicted in a line-shaped manner (for example FIG. 23).

Located opposite to the front face 53, an outer edge 54 of the transition region 52 is formed. This outer edge forms a further limitation of the buckling convex region 36. The outer edges extend approximately in the circumferential direction to the pressure region.

The buckling convex region 36 is concretely formed by a convexly formed bottom region, as already mentioned, between the front face and the outer edge, comprising a theoretical central axis of curvature, which extends on the inner side of the pump wall, and which, in turn, extends from the fastening edge in the direction of the pressure region. Preferably viewed from the fastening edge, a transition region 52 is formed so as to taper towards the pressure region, accompanied by a widening of the convex region 35.

More preferably, radially on the outside upstream of the fastening edge, the transition region 52 transitions into a surface, which is continuous in the circumferential direction. Preferably viewed from the fastening edge in the direction of the pressure region, a depth of the convex region 35 is initially also embodied so as to increase, with a largest depth approximately in the vicinity of the circumference or, viewed radially from the outside, just upstream of the buckling convex region, respectively. The largest depth can be provided approximately by 0.5-times to 2-times of a radial dimension of the buckling convex region upstream of a lowest position of the buckling convex region.

While a bottom of a convex region 35 runs continuously concavely, namely in a transverse as well as in a longitudinal direction, a bottom region of a buckling convex region, in contrast, runs convexly, almost in a saddle-like manner.

An air volume results between the pump wall 32 and the housing cover 24, which, in the course of a pump movement of the pump wall 32, in response to a movement from the initial position into the pump position, is subjected to a pressure and can serve for the compressed air blow-out of a chamber 26 as a result of a flow-related joining with the air guiding channel 14.

The space resulting between the pump wall 32 and the housing cover 24 is preferably joined to the air guiding channel 14 in the device housing 2 via perforations 37 with regard to the flow-related joining.

A convex region 35 has a convex edge 47. Edge points P1, P2, which, with regard to this convex edge 47, are spaced apart farthest away from one another (extension dimension E), see for instance FIG. 3, can be determined on the convex edge 47.

For an inclusion in a chamber 26 located in an emptying region 46, the air guiding channel 14 can have a deflection. The deflection can be reached by means of a blocking wall 38, which runs for example in a slide-like manner between two sections of the air guiding channel, which are embodied so as to correspond to one another in a longitudinal or flow direction, respectively, or parallel to one another, oriented transversely to these sections. As a result of the blocking wall 38 or of a corresponding embodiment of the air guiding channel 14, which is provided apart from that, the air flow can be forcibly guided again systematically through a replacement cover 39 of an activated chamber 26 and through the chamber 26 and the replacement cover 39 back into the air guiding channel 14. A chamber 26, which is hereby located in an emptying region 46, hereby forms a part of the air guide in this emptying region 46. A triple deflection of the air flow results in this region, initially by approx. 90° on an inflow side with regard to the chamber, then by approx. 180° through the chamber itself, and finally once again by approx. 90° to the return of the flow direction prior to the first deflection by approx. 90°.

More preferably, as follows for instance from FIG. 29, one or more or possibly also more further deflections are formed, until the final escape of the air, which is loaded with substance, through the discharge nozzle 15. Preferably, in relation to the mentioned cross section, initially in terms of an acute angle and subsequently possibly in terms of an obtuse angle.

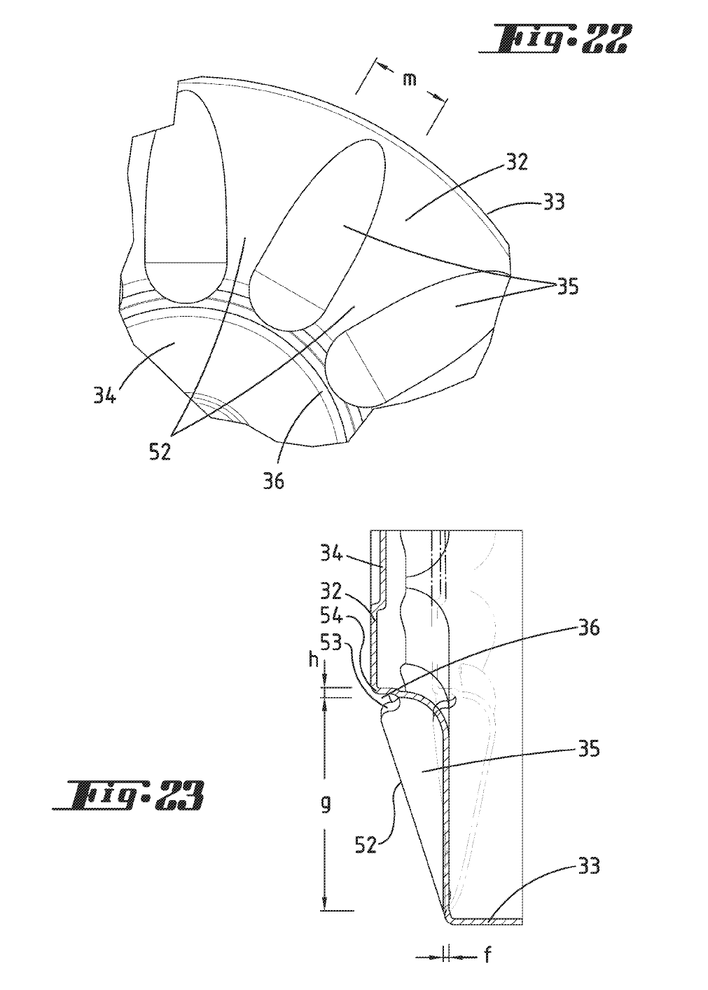

For this purpose, the replacement cover 39 is thus designed to be air-permeable, for example, as also illustrated, as a result of a grid-like embodiment according to the illustrations in FIGS. 15 and 16. In the exemplary embodiment, slits 42 are embodied in the cover, preferably comprising a longitudinal extension transversely to a flow direction of the air, which flows through.

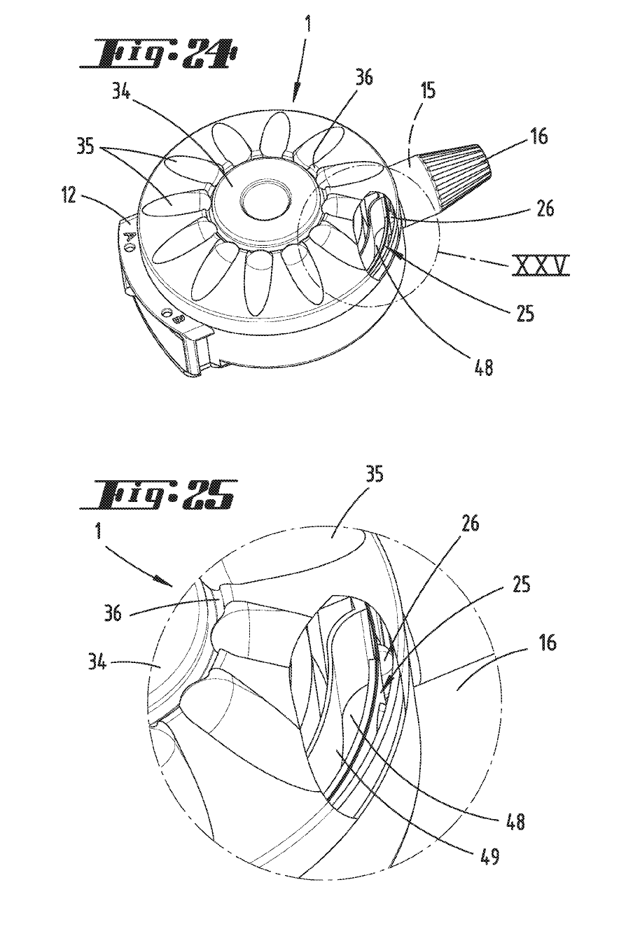

As follows in particular from FIGS. 15 to 17, the replacement cover 39 can be embodied integrally with the blocking wall 38 and a cover-side inlet opening 57. In this case, an insert part is formed, as illustrated in FIG. 17, which has a longitudinal axis, which runs perpendicular to the orientation of the blocking wall 38. In the direction of this longitudinal axis, the insert part can be inserted into a receptacle, which can be embodied in the device housing 2. The receptacle is preferably open on the side located opposite the housing bottom 3, for inserting purposes. As follows in particular from FIGS. 7, 8, 11, 12 and 29, the insert part can thus be inserted into the housing in the direction of the device axis x. For this purpose, a housing-side holding block 61, which forms a receptacle for the insert part and which has the mentioned receptacle, can be embodied.

As can be seen, the blocking wall 38 is formed with an arcuate layout on its end, which faces away from the replacement cover 39, for fitting into the air channel, which, moreover, is designed in this way there.

As follows in particular also from FIG. 15a, the blocking wall 38 is held on the ribs 58 or is embodied integrally therewith, respectively, on the side, which faces away from the chamber in the installed state. On the side facing away from the chamber, the ribs 48 can transition into the blocking wall 38 via foot regions 59, which protrude in a wedge-like manner.

From below, the replacement cover 39 covers the chamber 26, which is to be emptied and which is freed from the second layer 28, which serves as closure element (in the case of a mounting of the device with a vertical extending transversely to the device axis x and the mounting of the chamber in the emptying region “on the top”)—see also FIG. 8. In the case of another spatial mounting (holding by the user) of the device, a covering of the chamber 26 can result as well.

The replacement cover 39 prevents a trickling of the substance 27 out of the chamber 26 into the air guiding channel 14 solely dependent on the force of gravity. The replacement cover 39 replaces the second layer 28, but with a functionality, which differs with regard to a discharge of substance 27 from a chamber 26.

The transport of a chamber 26 into the emptying region 46 and thus into the inhalation-ready position according to for example the illustration in FIG. 7 or FIG. 8, respectively, preferably takes place by means of a back and forth pivot displacement of the transport lever 9, wherein an overrun of the toothed ring 19 by the latching fingers can initially be reached in response to a pivot displacement of the transport lever 9 from an initial position. So far, the transport wheel 22 does not move, is blocked as a result of the engagement of the retaining fingers 23 with the locking toothing 6.

As a result of the pivot return displacement of the transport lever 9 into the initial position, the transport wheel 20 can be entrained via the entraining between the latching finger 18 and the toothed ring 19, whereby the next chamber 26 is moved from a reservoir 45 into the emptying region 46 and thus into the inhalation-ready position according to FIG. 7, preferably by simultaneously removing the corresponding region of the second layer 28 or of the closure element, respectively, by means of peeling. The transport of the second layer as well can be accomplished solely by means of the removal movement of the first layer.

A continuous marking 40 is hereby preferably provided, which is visible for the user through a housing-side window 41. The markings 40 can be applied on the side of the first layer 43 of the strip-shaped element 25, which faces away from the second layer 28, as illustrated, for example in the form of numbers (see FIG. 10).

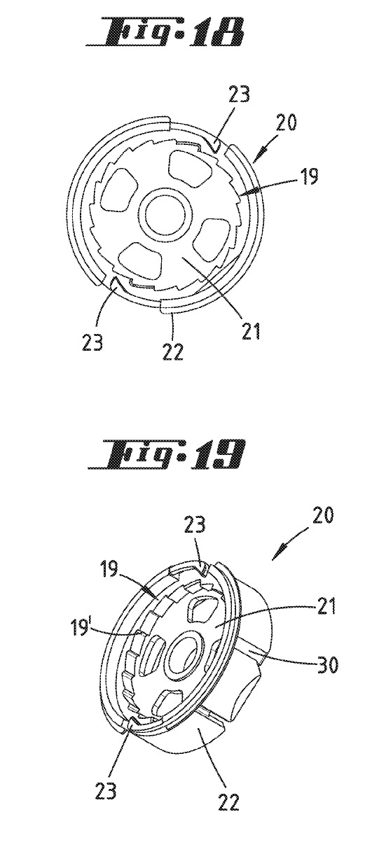

The chamber 26, which is in ready position, is part of the air guiding channel 14. In FIG. 12, an air flow, which evacuates the chamber 26, is illustrated schematically by means of the arrows u. With regard to the chamber, which is located in the emptying region 46, the part of the air guiding channel 14 mounted upstream of the chamber in the flow direction, connects virtually hermetically to the strip-shaped element 25, as also follows for instance from FIGS. 7 and 8. However, the air guiding channel 14, which is embodied in a housing-fixed manner, does not protrude beyond an opening plane 0 of a chamber into the latter.

In the case of the proposed device 1, the forced air flow is achieved by means of a surge-like pressurization. It is thus a compressed air blow-out of the chamber 26, after which the air, which is subjected to the forced flow, is loaded with substance 27, which is evacuated from the chamber 26. Preferably, a swirling of the substance in the air results as well. Swirled in the blow-out air in this way, the substance can be inhaled through the discharge nozzle 15.

The compressed air pressurization takes place by means of the bellows-like pump device 31, in response to which, upon operation by the user, a force threshold has to be overcome initially, as a result of the above-described convex embodiments in the region of the pump wall 32. They work in the manner of a clicker, so that a blow-through of the chamber 26, which starts almost abruptly, takes place.

As a result of the force threshold, which needs to be overcome initially, it is ensured that a sufficiently high flow speed for the complete evacuation of the chamber 26 is reached.

To overcome the force threshold in response to the operation of the pump device 31, a force introduction essentially in the direction of extension of the axis x radially to the inside of the device housing 2 amounting to several Newtons is required. The hereby triggered impact can lead to a significant air flow speed, in particular in the region of the chamber 26 to be evacuated. Such a speed and also the created amount of air are insufficient to safely and completely empty the chamber 26 and to remove the substance 27, in particular the powdery medicament, through the replacement cover 39.

The high air flow speed preferably also results from a large ratio of the effective surface of the pump wall to a cross section of the air guiding channel. The cross section of the air guiding channel outside of the replacement cover corresponds to one-tenth or less, up to one-twentieth, one-fiftieth or one-hundredth or even less, of the mentioned effective surface of the pump wall. This effective surface is significantly reduced once again in the region of the replacement cover, once again by half or more, up to one-tenth or one-hundredth, as compared to the region upstream of the replacement cover, reduced with regard to the effective opening, which controls the air inlet into the chamber.

The air preferably flows through the chamber 26 in the direction of its longitudinal extension. More preferably the air flows in the direction of the width of the first layer, when it flows through a chamber 26.

Provided that an open chamber 26 and thus the chamber, which is ready for inhalation, is not used (as a result of an omission of the inhalation process by a user), the content is possibly unloaded into a resulting region, which cannot be joined to the air guiding channel 14, in response to one of the next advancing movements for moving a next chamber into the ready position, or the substance 27 can trickle into this region, respectively, a receiving region. This region is preferably the receiving chamber 48, which will also be described below in further detail.

With reference to in particular FIGS. 24 to 27 and 30, the receiving chamber 48 is described in further detail.

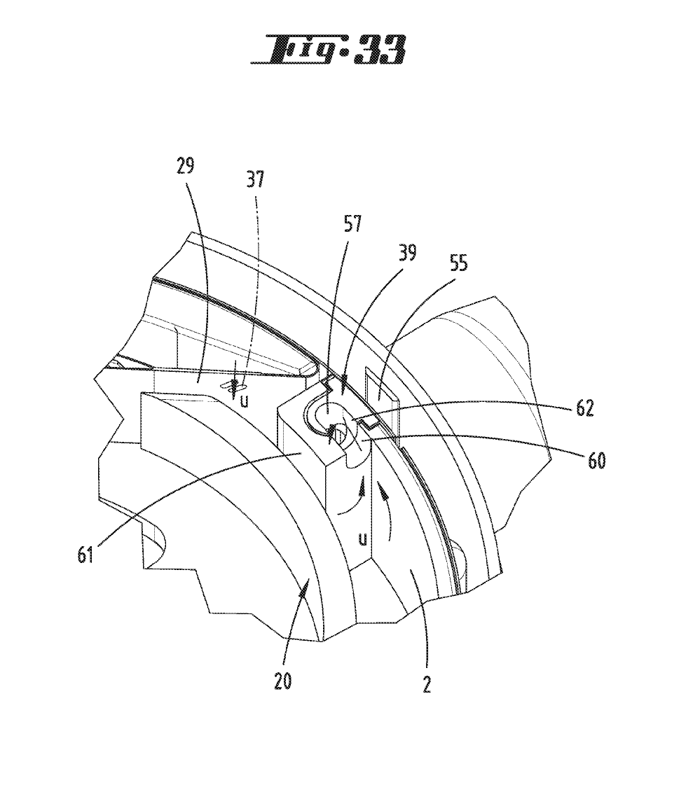

The receiving chamber 48 is initially formed on the bottom and cover side by the housing cover 24 and the housing bottom 3. It is preferred hereby that the housing cover 24 is in vertical tight abutment with a wall 49, which protrudes from the housing bottom 3, also over a partial height (extension in the thickness direction of the device) of the receiving chamber 48 for the sealing cooperation, see in particular FIG. 30, the section of the sealing abutment k.

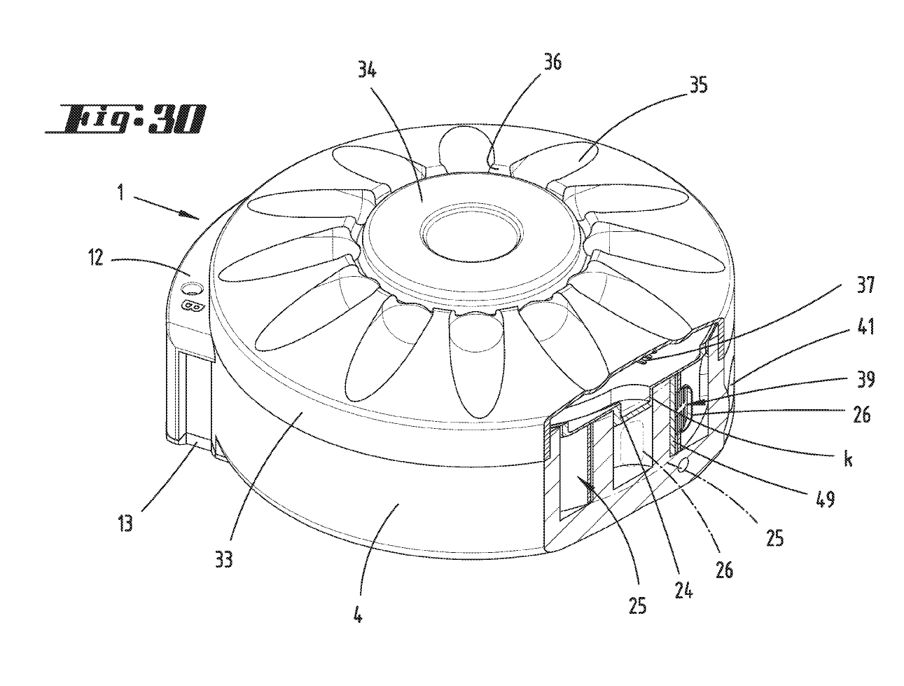

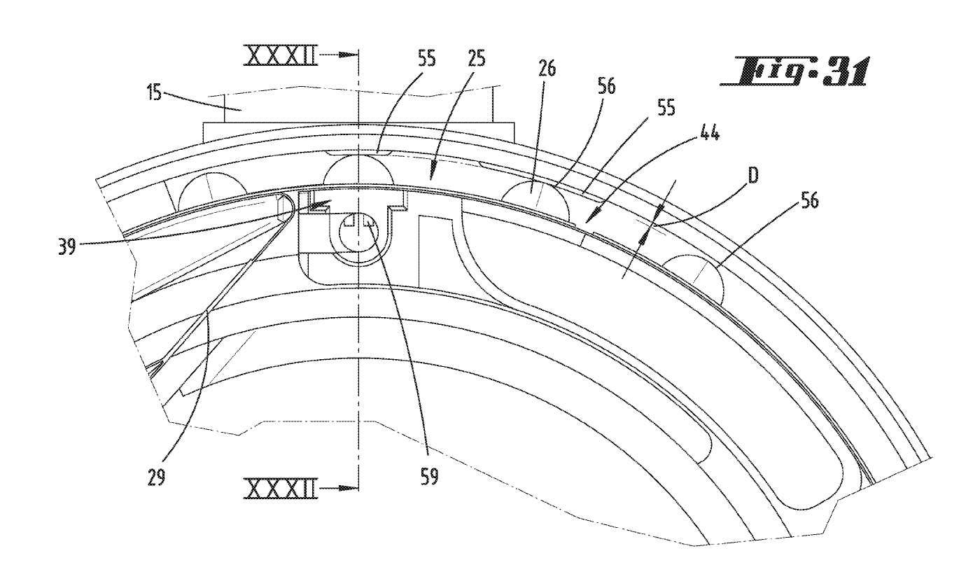

The wall 49 can be embodied circumferentially, for the purpose of which reference can for instance also be made to FIGS. 25 and 27. It is important hereby that the wall 49 leaves open a window 50, in which an open chamber 26 is exposed with regard to its opening plane towards the receiving chamber 48. Substance, which is perhaps still located in the chamber 26, can thus trickle into the receiving chamber 48.

As a result of a preferably tight abutment of the strip-shaped element and in particular of the partial region, with the open chamber 26, on the outside of the wall 49, which is assigned to the receiving chamber 48 within the given time period, it is ensured that no substance can penetrate from the receiving chamber 48 into the housing apart from that. For this purpose, a certain pressure application of the strip-shaped element on the outer surface of the wall 49 is advantageous. It is in particular preferred that the partial region with the chamber 26, which is located in the window 50, has a contact surface on the outside circumferentially to the window so as to be closed circumferentially.

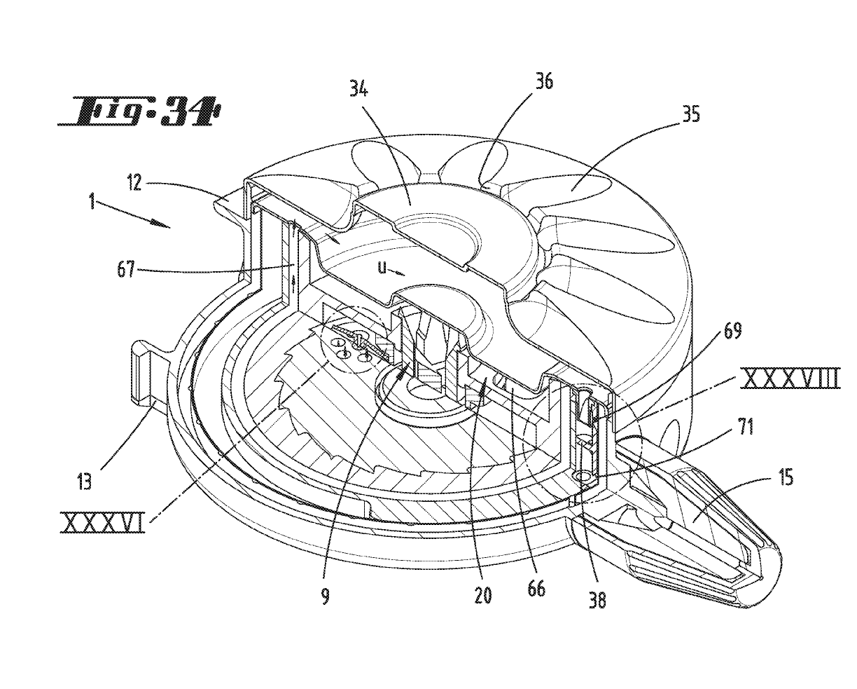

With reference to FIGS. 31 and 32, the pressure application can be formed by means of a thickened embodiment by the measure D of a guide wall 55, against which a chamber elevation 56 comes to rest. In the alternative, it can also be formed by means of only one rib, which then preferably extends approximately centrically to the length of a chamber on the guide wall 55 in the circumferential direction. The latter has the advantage of a smaller impact on the required transport force. The film-like design of the first layer also results in a virtually elastic pressing effect of the front side of the chamber, in the opening plane, in the emptying region or also in the region of the receiving chamber.

The mentioned thickening of the guide wall or the mentioned rib is preferably also embodied only over the circumferential region of the guide wall 55, which corresponds to the extension from the emptying region to a positioning of a chamber on the window of the receiving chamber. Evidently in each case also beyond that to a certain extent, as can be seen from FIG. 31.

A blocking wall 38, which is suitably embodied on the replacement cover 39, divides the chamber approximately in the center into an inflow and an outflow region with regard to the air, which permeates the air guiding channel 14. With the exception of more preferably the front face of the blocking wall 38 itself, both regions are also divided by the replacement cover 39 against the chamber 26 or a corresponding opening plane, respectively.

As a result of the created forced flow, air, which is loaded with substance, can permeate the replacement cover 39, and then evidently preferably initially flows out of the chamber in the opposite direction, but separated by the blocking wall 38, of the air, which flows into the chamber 26 and which is not loaded with substance, and experiences a further deflection, downwards in FIG. 7, so as to then enter into the discharge nozzle 15 as a result of a further, more preferably also acute-angled deflection, and to then escape therefrom, for instance into a nose or a mouth of a user. As is also already done by the permeation of the replacement cover 39, the mentioned repeated deflections of the air, which is loaded with substance, additionally ensure a good mixing and breaking open of agglomerations of substance, which may still be present. As soon as the user then ends the force application of the pump wall, the pump wall 32 returns automatically and air is inhaled again into the space between the pump wall 32 and the housing cover 24 through the discharge nozzle 15 in the opposite direction.

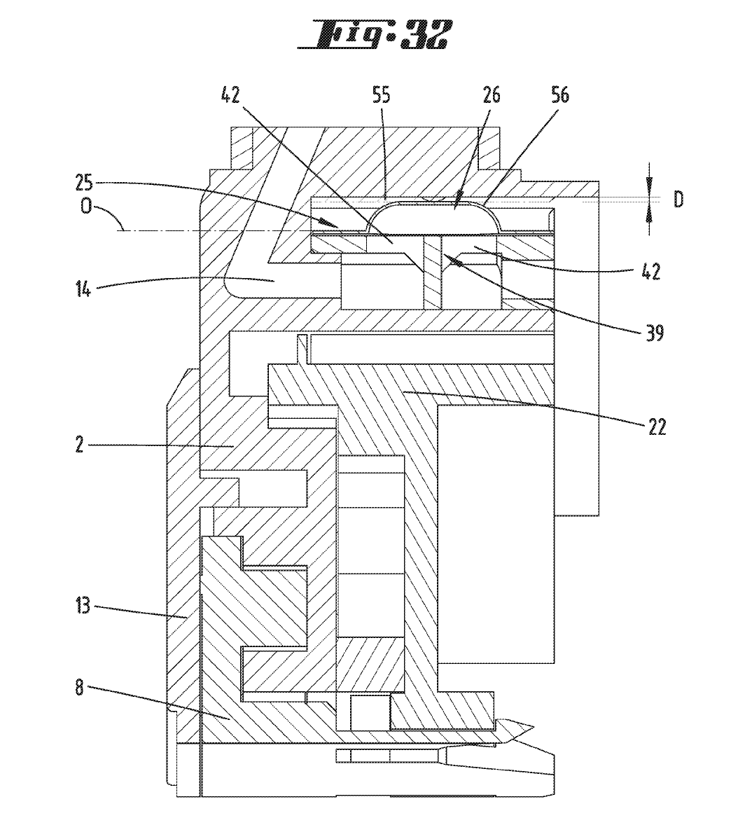

The space between the pump wall 32 and the housing cover 24 can have a further outlet or passage for air into the region of the housing located therebelow, see perforation 51 for instance in FIGS. 28, 29. In response to a movement of the pump wall 32 in the pump direction as well as in the aspiration direction, air flows through the perforation 51 in the same direction as to the perforation 37, wherein air, which flows through the perforation 51, preferably accounts for only a significantly smaller portion. On principle, the perforation 51 can also be forgone.

According to the illustrations in FIGS. 28, 29 and 33, air flows through the perforation 37 (or a plurality of perforations), which is reproduced in FIG. 33 with a dash-dotted line type in response to an operation of the pressure region 34, into a space, which results between the housing 2 and the transport wheel 20 and into which the holding block 61 comprising the replacement cover 39 protrudes on the side of the mouthpiece. The inlet opening of the replacement cover 39 can be covered by the housing cover 24 (see for example FIG. 28) in this embodiment.

A flow-related joining can be achieved by embodying a transverse bore 60 in the holding block 61, which transitions into a puncture channel 62 of the replacement cover 39. The puncture channel 62 is directed transversely to the inlet opening 57 and runs into the latter.

A deflection of the air flow u by approximately 90° results within the replacement cover 39 before the air enters into the inlet opening 57.

In the alternative, the air flow u can also run essentially in the same direction as a geometric central axis of the inlet opening 57 prior to passing through the inlet opening 57. In this case, the perforation 37 is mounted directly above the holding block 61, in particular directly above the replacement cover 39 (see for example FIG. 8).

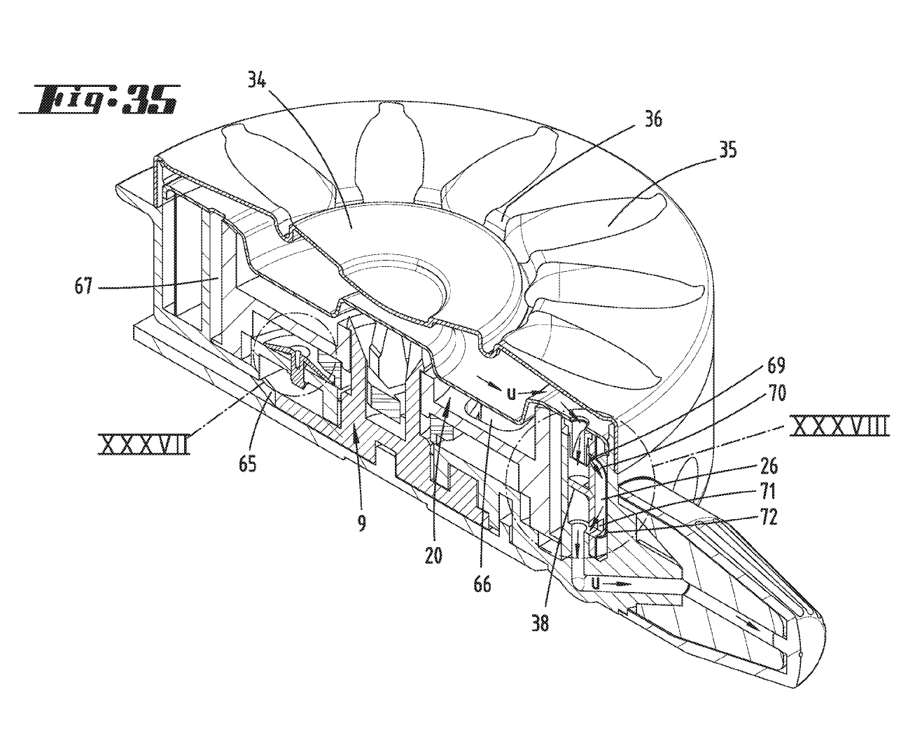

With regard to FIGS. 34 to 37, an embodiment is illustrated, in the case of which the aspiration of air in response to the returning of the pump wall takes place on a separate airway. Such an embodiment can also be realized in the case of the embodiments, which have already been described above. A partial back-suctioning through the outlet nozzle and the air guiding channel and a partial back-suctioning via the separate airway can hereby take place as well.

With regard to the separate airway, it is provided in detail that one or more through openings 63 are embodied in the housing bottom 3, see also FIG. 9, preferably in the region of the section, which results in the locking toothing 6. The one or more through openings 63 can be covered by a non-return valve 64. The non-return valve 64 opens in response to an aspiration of the air and closes, when air is to be discharged in a pump movement through the air guiding channel 14.

The air can have reached to the through openings 63 through a passage opening 65, which can remain between the transport lever 9 and the housing bottom 3.

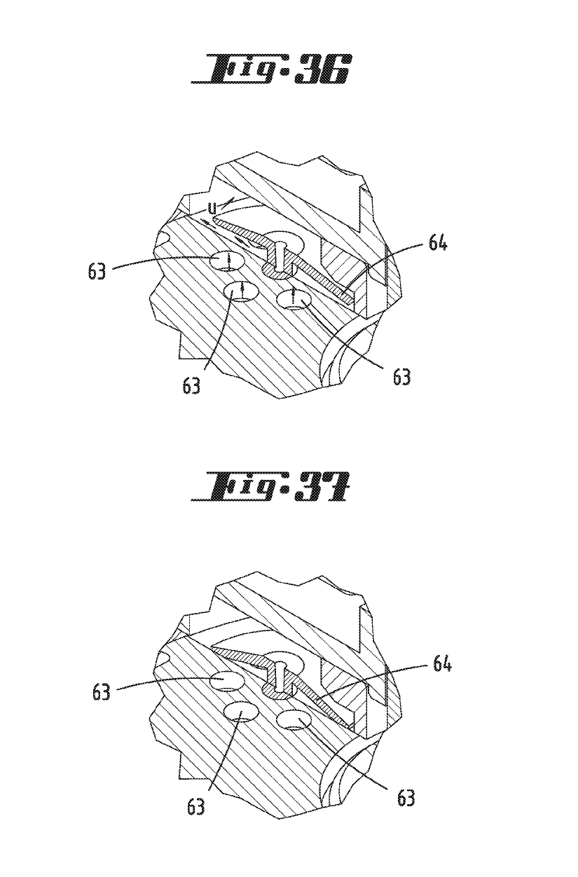

The intake air, which passes through the through openings 63, see FIG. 36, can enter via openings 66 in the bottom 21 and openings, which remain between the housing cover 24 and the bottom 21, into the space between the housing bottom 3 and the bottom 21 of the transport wheel 20, and can enter via said space by means of the perforation 51 into the space between the housing cover 24 and the pump wall 32.

In the case of this embodiment, it is preferred with regard to an air flow, which serves to evacuate a chamber 26, that said air flow permeates a non-return valve 68 and/or a screen 67 in flow direction upstream of the chamber 26. In the case that no or no sufficiently effective replacement cover 39 is embodied, the non-return valve 68 has the particular significance of preventing that substance could reach with a return flow into the region upstream—based on the air flow, which serves to evacuate the chamber 26—in particular between the pump wall and the housing cover. A return flow can be reliably prevented by means of the non-return valve 68.

In further detail, the non-return valve 68 can be embodied for instance as lip valve. In the non-pressure state, the lips abut against one another so as to form a seal and are only pressed against each other in response to a back-suctioning. In contrast, this air can pass through by simply spreading the lips away from one another in response to a permeation with compressed air in the direction of the chamber 26.

A screen 67 can already offer a certain amount of help as well. In response to a back-suctioning, whereby the screen 67 can also be combined with an embodiment, in the case of which the back-suctioning takes place completely or partially through the discharge nozzle 15, particles, which may be located in the back-suctioning stream, can be effectively prevented from entering into the space between the pump wall and the housing cover by means of the screen 67.

In further detail, a mounting recess for the non-return valve 68 and/or the screen 67 can be provided in the insert part, which has the replacement cover 39. Together with the replacement cover 39, the non-return valve 68 and/or a screen 67 can thus be mounted easily.

In connection with the embodiments described last here, thus in the case of which a screen 67 and/or a non-return valve 68 is provided, but also for use in the embodiments described above, it is also preferred that the replacement cover 39 does not have multiple slits or a screen structure with regard to the cover of the chamber 26 itself, see also FIG. 35, but only leaves a (at least) first air passage 69, assigned to a chamber beginning 70, and a second air passage 71, assigned to a chamber end 72. In the case of this embodiment, the replacement cover assigned to the chamber 26 is moreover preferably embodied so as to be completely closed, with the exception of the mentioned air passages.

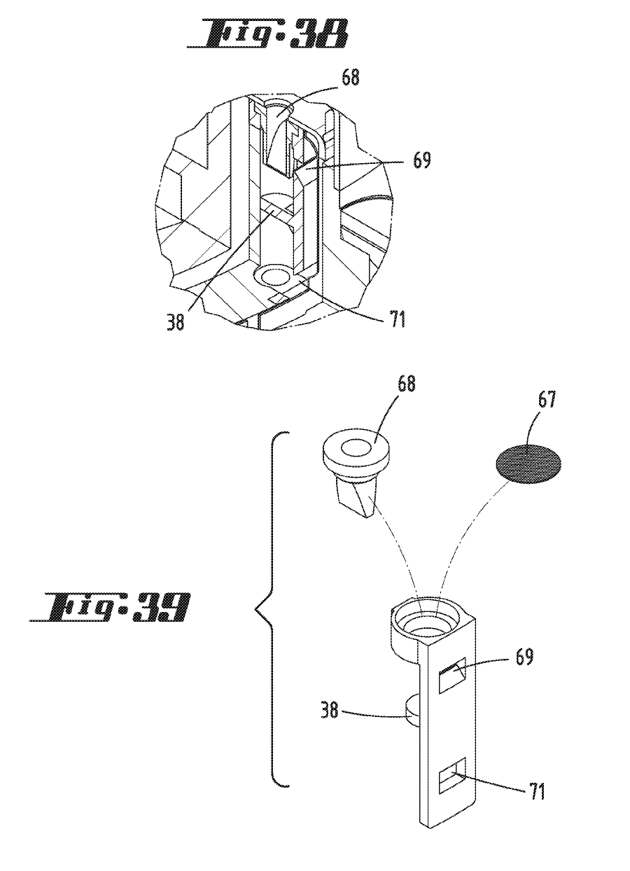

A corresponding enlarged illustration of the installation can also be gathered from FIG. 38.

Such an embodiment of the replacement cover can also be provided in the case of the embodiments as described above.

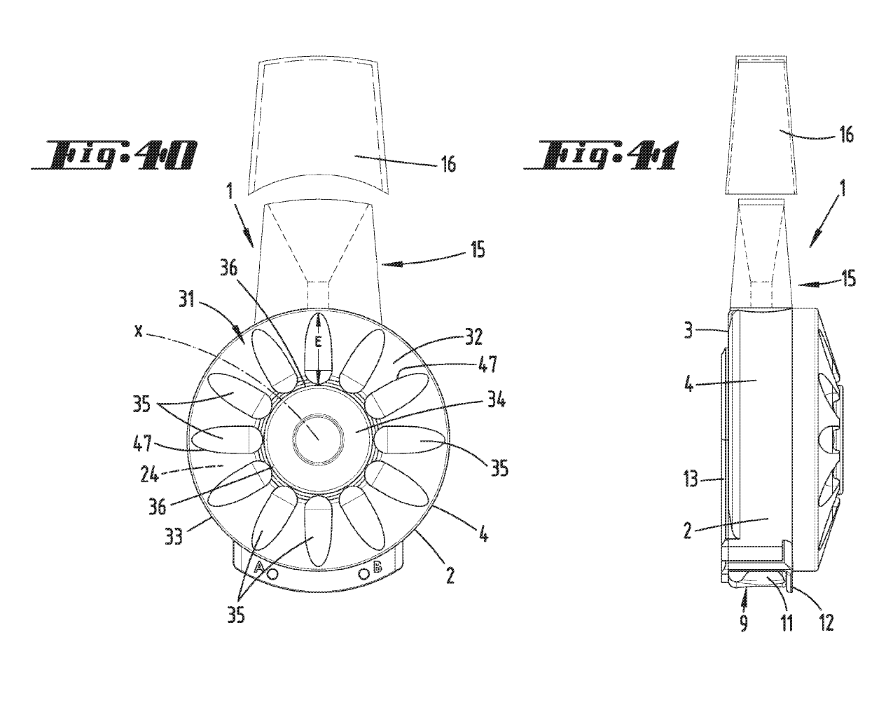

In the case of the embodiment of FIGS. 40 and 41, an identical embodiment is provided, as it corresponds to the above-described embodiments. Only the discharge nozzle 15 is embodied here as mouthpiece. It serves to be used for inhaling by breathing in via the mouth of a user. The large width resulting from the top view according to FIG. 40 as compared to a small thickness, as it can be gathered from FIG. 41, is essential.

With reference to FIGS. 28 and 29, and in particular also FIGS. 1, 2, 7, 8, 12, 23 and 32, the following results in response to a use:

In an initial state, the device for instance according to FIG. 1 is provided for a user. The strip-shaped element 25 is located in the device 1 and is inserted through the slit formation 30 by means of the handling section 29 and is thus caught on the transport wheel 20.

A person using the device initially removes the cap 16 from the discharge nozzle 15 and then moves the operating section 11 in the region of the boundary wall 12 by a possible angular amount and back again. A partial region of the strip-shaped element 25 is thereby moved out of the reservoir 45 into the emptying region 46, so that a chamber 26 is located in the position according to FIG. 12 or FIG. 32, respectively. As a result of its fastening to the transport wheel 20 by means of the handling section 29 in the course of the movement, the second layer 28 has been removed from the chamber 26, which is then located in the emptying region 46.

In detail, the joining pins 8 thereby rotate the follower 17, which is joined thereto in a rotationally fixed manner, which, in turn rotates the transport wheel by a corresponding angular amount by means of the impact on the teeth 19′ of the toothed ring 19 of the transport wheel 20. On the one hand, the second layer 28 is hereby removed from the first layer 43 by means of the handling section 29, which is formed from the second layer 28, and the (first) chamber 26, which is exposed accordingly in the opening plane 0 as a result of the removal of the second layer 29, is simultaneously transferred into the emptying position according to FIG. 11 or FIG. 12, respectively. In response to the backward movement of the operating section 11 and thus of the lever arm 10, the latching fingers 18 run over the teeth 19′, because the transport wheel 20 is prevented from a return movement as a result of the retaining fingers 23, which engage with the locking toothing 6 of the housing bottom 3.

In the case of the special embodiment, the chamber 26 is then covered by the replacement cover 39 in the emptying region 46. A user then needs to surround the discharge nozzle 15 with his mouth. As a result of the pressurization of the pressure region 34, the user can subsequently move the pump wall from the initial position into the pump position (see FIGS. 8 and 11, 12) and can thereby create an unstoppable and comparatively large and strong air flow as a result of the described invasion effect.

The air flow flows from the region between the pump wall 32 and the housing cover 24 through the perforation 37 into a first region of the air guiding channel 14, which leads all the way to the strip-shaped element 25. The air guiding channel is then continued by the chamber 26, which is to be emptied, preferably by interpositioning and also by flowing through the replacement cover 39. The substance from the chamber 26 is thereby entrained.

After flowing through the chamber 26, the air flow, now loaded with substance, passes through a further section 14 of the air guiding channel and into the discharge nozzle 15 under sharp deflection, and thus reaches into the mouth of the user.

Immediately after releasing the pressure load by the user, the pump wall 31 returns back into the initial position. The device is ready for a further use.

REFERENCE LIST

| | | | |
|---|---|---|---|
| 1 | device | 29 | handling section |
| 2 | device housing | 30 | slit formation |
| 3 | housing bottom | 31 | pump device |
| 4 | housing wall | 32 | pump wall |
| 5 | support wall | 33 | fastening edge |
| 6 | locking toothing | 34 | pressure region |
| 7 | bore | 35 | convex region |
| 8 | joining pin | 36 | buckling convex region |
| 9 | transport lever | 37 | perforation |
| 10 | lever arm | 38 | blocking wall |
| 11 | operating section | 39 | replacement cover |
| 12 | boundary wall | 40 | marking |
| 13 | counter plate | 41 | window |
| 14 | air guiding channel | 42 | slit |
| 15 | discharge nozzle | 43 | first layer |
| 16 | cap | 44 | element end |
| 17 | follower | 45 | reservoir |
| 18 | latching finger | 46 | emptying region |
| 19 | toothed ring | 47 | convex edge |
| 20 | transport wheel | 48 | receiving chamber |
| 21 | bottom | 49 | wall |
| 22 | wall | 50 | window |
| 23 | retaining finger | 51 | perforation |
| 24 | housing cover | 52 | transition region |
| 25 | strip-shaped element | 53 | front face |
| 26 | chamber | 54 | outer edge |
| 27 | substance | 55 | guide wall |
| 28 | second layer | 56 | chamber elevation |
| 57 | inlet opening | a | length |
| 59 | rib | b | width |
| 59 | foot region | c | width |
| 60 | transverse bore | d | width |
| 61 | holding block | e | thickness |
| 62 | puncture channel | f | thickness |
| 63 | through opening | g | length |
| 64 | non-return valve | h | length |
| 65 | passage opening | m | width |
| 66 | opening | u | air flow |
| 67 | screen | x | device axis |
| 68 | non-return valve | D | measure |
| 69 | air passage | E | extension dimension |
| 70 | chamber beginning | F | surface |
| 71 | air passage | O | opening plane |
| 72 | chamber end | P1 | point |
| | | P2 | point |
| | | R | longitudinal chamber |
| | | | direction |

What is claimed is:

1. A device for dispensing a substance, which can be discharged by air, comprising:
a discharge nozzle, through which air, which is loaded with substance, can escape,
a pump device comprising a pump wall, which forms a part of a surface of the device and which can be operated by hand to create air under forced flow,
an air guiding channel joining the pump device to the discharge nozzle, through which the air, which is subjected to the forced flow, can be guided,
a strip-shaped element, which is mounted in the device and which has a length and a width, wherein the length is several times larger than the width,
chambers, which are embodied successively over the length in the strip-shaped element and in which the substance is received,
a reservoir, in which the strip-shaped element can be received,
an emptying region, in which a partial region of the strip-shaped element can be moved out of the reservoir in the direction of the length, for discharging substance from a chamber by means of the air subjected to the forced flow,
wherein the strip-shaped element has a first layer and a second layer, which layers are aligned with each other and run so as to overlap one another,
wherein a plurality of chambers, to which a partial region is assigned in each case, is embodied in the first layer, the chambers are protected by the second layer against a loss of substance, and the second layer can be removed from the first layer whereupon the chamber is exposed in an opening plane, which corresponds to a joining plane between the first and the second layer,
wherein in the case of a chamber located in the emptying region, the second layer is already removed from the partial region having this chamber and the air subjected to the forced flow flows through the air-guiding channel merging into the chamber in the opening plane so as to be directed into the chamber, and
wherein the chamber is moved in an inhalation-ready position downstream from an air-permeable replacement cover.

2. A device for dispensing a substance, which can be discharged by air, comprising:
a discharge nozzle, through which air, which is loaded with substance can escape,
a pump device for creating air subjected to a forced flow, the pump device having a pump wall, which can be moved between an initial position and a pump position and which can be automatically returned from the pump position into the initial position, and
an air guiding channel, which joins the pump device to the discharge nozzle and through which the air subjected to the forced flow can be guided,
wherein the pump wall has a fastening edge, by means of which the pump wall is fastened to a housing of the device so as to form a seal, a pressure region, and several convex regions outside of the application region, and
wherein the pressure region can be moved to carry out a pumping process by reducing an air volume located between the pump wall and the device housing, and the convex regions remain essentially undeformed in response to a movement of the pump wall from the initial position into the pump position.

3. A device for dispensing a substance, which can be discharged by air, comprising:
a discharge nozzle, through which air, which is loaded with substance can escape,
an air guiding channel, through which an air subjected to a forced flow can be guided,
a strip-shaped element, which is mounted in the device and which has a length and a width, wherein the length is several times larger than the width,
chambers, which are embodied successively over the length in the strip-shaped element and in which the substance is received,
a reservoir, in which the strip-shaped element can be received,
an emptying region, into which the strip-shaped element can be moved gradually by a partial region, in which a chamber is embodied, out of the reservoir in the direction of the length, for discharging substance from a chamber by means of air subjected to the forced flow, and
a receiving region, into which the partial region can be moved directly downstream from the emptying region in response to a following gradual movement,
wherein the receiving region is configured as a closed receiving chamber within the device and the partial region, which had been located in the emptying region in response to the preceding movement, and forms a movable part of a wall of the receiving chamber, which cooperates with stationary parts of the wall of the receiving chamber so as to form a seal, and
wherein the strip-shaped element is guided so as to abut on corresponding stationary wall regions of the receiving chamber so as to form a seal on the edge.

4. The device according to claim 2, wherein a convex region has a convex edge.

5. The device according to claim 2, wherein a convex region is formed from the pump wall itself with essentially a same thickness as the pump wall outside of a convex region.

6. The device according to claim 5, wherein each of the convex regions has a largest extension dimension measured between two edge points of its convex edge located farthest away from one another, and the thickness of the pump wall is one-tenth or less of the largest extension dimension of a convex region.

7. The device according to claim 2, wherein a convex region is embodied so as to be elongated, comprising a longitudinal direction, a length and a width, wherein the longitudinal direction runs from the fastening edge in the direction of the pressure region.

8. The device according to claim 7, wherein a convex region remains uninfluenced with regard to its length and width in response to a movement in the course of a pumping process.

9. The device according to claim 7, wherein two convex regions are arranged next to one another in the longitudinal direction of a convex region.

10. The device according to claim 2, wherein the pump wall is fastened with the fastening edge to a device housing so as to form a seal and wherein the pressure region can be moved to carry out a pumping process by reducing an air volume located between the pump wall and the device housing.

11. The device according to claim 2, wherein a buckling of the pump wall occurs in a buckling convex region in response to a pump movement, wherein the convex region and the buckling convex region have different lengths, wherein the buckling convex region has a smaller length than the convex region, and wherein the smaller length corresponds to 1/50 to ⅓ of the larger length of the convex regions, which are embodied one behind the other.

12. The device according to claim 1, wherein the air, which is directed to the chamber and is subjected to the forced flow, experiences a deflection by means of a chamber wall.

13. The device according to claim 1, wherein the chamber of the partial region, which is located in the emptying region, of the strip-shaped element forms a part of the air guiding channel.

14. The device according to claim 1, wherein one of the chambers has a length and a width, wherein the length is larger than the width and wherein the length of the chamber is oriented transversely to a length of the strip-shaped element.

15. The device according to claim 14, wherein the air flows through the chamber, when it forms a part of the air guiding channel, in the direction of its length.

16. The device according to claim 2, wherein
the device housing has a first and a second broad side, which are located opposite one another with outer surfaces formed so as to face away from one another and which are essentially congruent in a top view onto one of the broad sides and are spaced apart by a narrow side,
wherein the first and the second broad side further have a corresponding smallest width dimension, which is measured along a line through a center of an area of the broad side based on the top view and wherein the narrow side determines a thickness of the device housing,
wherein the pump wall furthermore forms a part of the first broad side, the thickness is smaller than the smallest width dimension, and the discharge nozzle, viewed in the top view, appears as a formation of the broad side, which protrudes in a tip-shaped manner,
wherein the pump wall is further fastened with the fastening edge to a device housing so as to form a seal, the pressure region can be moved to carry out a pumping process by reducing an air volume located between the pump wall and the device housing, and a buckling of the pump wall into a buckling convex region takes place in response to a pump movement,
wherein the device has a transport device for transporting a chamber into an initial position, wherein an operable transport lever for a user protrudes on a narrow side of the device housing,
wherein the transport lever is arranged on the device housing located opposite to the discharge nozzle, and/or
wherein the device housing has a basic contour, which is circular viewed in pressure direction of the pump wall, wherein the discharge nozzle and/or the transport lever protrudes beyond the basic contour.

17. The device according to claim 16, wherein the transport lever is surrounded by a boundary wall, which also protrudes radially beyond the circular basic contour.

18. The device according to claim 2, wherein
the pump wall further has a fastening edge, a pressure region and several convex regions, which are embodied so as to surround the pressure region in a circumferential direction and which are arranged outside of the pressure region and between the fastening edge and the pressure region, and the pump wall as a whole is embodied so as to be closed, and
wherein the convex regions maintain their shape in response to a movement of the pump wall by the impact of force on the pressure region from the initial position into the pump position, in the case of which the fastening edge can be unmoved.

19. The device according to claim 18, wherein, viewed in a circumferential direction, rib-like transition regions extend between the convex regions between the fastening edge and the pressure region.

20. The device according to claim 19, wherein one of the transition regions has a front face, which faces the pressure region, wherein the pressure region has an outer edge located opposite the front face and running in the same direction and wherein in the pump position, a buckling of the pump wall occurs between the front face and the outer edge.
